(19) United States
(12) Patent Application Publication
(10) Pub. No.: US 2021/0338716 A1
(43) Pub. Date: Nov. 4, 2021

(54) Sodium Chlorite Compositions with Enhanced Anti-Viral and Anti-Microbial Efficacy and Reduced Toxicity

(71) Applicant: iRenix Medical, Inc., Palo Alto, CA (US)

(72) Inventors: Stephen J. Smith, Palo Alto, CA (US); Thomas W. Chalberg, JR., Palo Alto, CA (US)

(21) Appl. No.: 17/245,675

(22) Filed: Apr. 30, 2021

Related U.S. Application Data
(60) Provisional application No. 63/018,057, filed on Apr. 30, 2020.

(51) Int. Cl. A61K 33/20; A61J 1/20; A61K 9/00; A61K 47/02; A61K 47/12; A61P 27/02; A61L 2/08; B65D 81/32

(52) U.S. Cl. CPC A61K 33/20; A61J 1/2089; A61K 9/0048; A61K 47/02; A61L 2202/23; A61P 27/02; A61L 2/087; B65D 81/3255; A61L 2202/18; A61K 47/12

(57) ABSTRACT

Methods of treating a subject for a microbial eye condition are provided. Aspects of the methods include administering to the subject an activated sodium chlorite composition, where the compositions include sodium chlorite; and a buffer component prepared from sodium phosphate monobasic monohydrate and citric acid. Also provided are methods of inhibiting a virus associated with a tissue, such as an adenovirus or coronavirus. In addition, delivery devices for administering an activated sodium chlorite composition to a tissue are provided.

### CROSS-REFERENCE TO RELATED APPLICATIONS

Pursuant to 35 U.S.C. § 119 (e), this application claims priority to the filing date of U.S. Provisional Patent Application Ser. No. 63/018,057 filed Apr. 30, 2020, the disclosure of which application is incorporated herein by reference in its entirety.

### INTRODUCTION

Ocular infections are a significant cause of morbidity and mortality globally. Viral conjunctivitis in particular is very common and extremely contagious. It has been estimated that up to 1-2% of primary care visits in the United States are secondary to viral conjunctivitis, and the cost of diagnosis and treatment of this infection is estimated to exceed $400 million. However, an even greater cost of these infections is reflected in lost productivity. Viral conjunctivitis frequently affects children, requiring them to be pulled out of day care and school, and consequently requiring parents and caregivers to take time off from work.

To date, there are no effective treatments for viral conjunctivitis, and this remains a key unmet need in ophthalmology. In addition, there are numerous other pathogens that result in ocular and non-ocular infections. The treatment of these is frequently limited by narrow spectrum treatment, and emerging strains that are resistant or become resistant to known treatments.

Another issue of great national and international importance is emerging infections with the potential to infect the population on a global scale, including the SARS-CoV-2 infection that emerged in China in 2019 and subsequently spread across the world in 2020.

Developing effective treatment strategies for existing and still yet to arise infections is of utmost priority, and the compositions disclosed herein have extremely broad anti-microbial, including anti-viral, properties coupled with excellent safety data.

### SUMMARY

Methods of treating a subject for a microbial eye condition are provided. Aspects of the methods include administering to the subject an activated sodium chlorite composition, where the compositions include sodium chlorite; and a buffer component prepared from sodium phosphate monobasic monohydrate and citric acid. Also provided are methods of inhibiting a virus associated with a tissue, such as an adenovirus or coronavirus. In addition, delivery devices for administering an activated sodium chlorite composition to a tissue are provided.

Implementations disclosed herein include an antiseptic composition for disinfecting tissues, the composition including sodium chlorite. The sodium chlorite can be in an amount of about 5 ppm to about 20,000 ppm, such as 100 ppm to 10,000 ppm, e.g., 4,000 ppm to 6,000 ppm. The sodium chlorite can be activated in a buffer having a pH that is less than or equal to 5 or up to about 10. The composition can further include a surfactant. The surfactant can be a non-ionic surfactant in an amount of between 0.015% w/v to about 1.0% w/v. The non-ionic surfactant can be one or more of polyoxyethylene sorbitan monooleate, polyoxyethylene lauryl ether, or poly(ethylene glycol)-block-poly(propylene glycol)-block-poly(ethylene glycol). The composition can have antimicrobial, anti-fungal, anti-viral, and anti-parasitic activity. The composition can be in a form including aqueous solutions, emulsions (oil-in-water or water-in-oil), lotions, creams, ointments, salves, gels, instillations, foams, powders, tinctures, and solids. The composition can be in the form of an eye drop, eye wash, eye swab, or an eye bath. The composition can be in the form of a solution, suspension, gel, swab, or bath for skin application. The tissues disinfected can include skin, eye, wound, or incision. The tissues disinfected can include skin, mucocutaneous membranes, mucous membranes, nasopharynx, lungs, eye lid, eyebrow, cheek, cornea, conjunctiva, or palpebral fornix.

In an interrelated aspect, disclosed are uses of a composition for the preparation of a medicament for the disinfection of tissues. The composition includes sodium chlorite activated in a buffer. The sodium chlorite can be in an amount of about 5 ppm to about 20,000 ppm, such as 100 ppm to 10,000 ppm, e.g., 4,000 ppm to 6,000 ppm. The sodium chlorite may comprise PURITE® 2% sodium chlorite stock solution. The buffer can have a pH that is less than or equal to 5 or up to about 10. The buffer can comprise sodium phosphate monobasic monohydrate 0.25% w/v, citric acid monohydrate 0.35% w/v and water. The combined sodium chlorite and buffer composition can have a pH between 3.0 and 4.5, such as 3.2 to 4.4, or about 4. The buffer can comprise sodium phosphate monobasic monohydrate 0.83% w/v, citric acid monohydrate 0.17% w/v, sodium hydroxide 1N 0.092% w/v and water. The combined sodium chlorite and buffer composition can have a pH between 4.2 and 5.5, or about 5. As such, the combined sodium chlorite and buffer composition, i.e., the activated sodium chlorite composition, can have a pH between 3.3 to 7.5, such as 3.0 to 4.5, 3.2 to 4.4, or about 4, or in other instances 4.5 to 5.5, or about 5. The composition can have antimicrobial, anti-fungal, anti-viral, and anti-parasitic activity. The composition can be in a form including aqueous solutions, emulsions (oil-in-water or water-in-oil), lotions, creams, ointments, salves, gels, instillations, foams, powders, tinctures, and solids. The composition can be in the form of an eye drop, eye wash, eye swab, or an eye bath. The composition can be in the form of a solution, suspension, gel, swab, or bath for skin application. The tissues disinfected can include skin, eye, wound, or incision. The tissues disinfected can include skin, mucocutaneous membranes, mucous membranes, nasopharynx, lungs, eye lid, eyebrow, cheek, cornea, conjunctiva, or palpebral fornix.

In an interrelated aspect, disclosed are methods of treating tissues including topically applying an antiseptic composition comprising sodium chlorite activated in a buffer. The sodium chlorite can be in an amount of about 5 ppm to about 20,000 ppm, such as 100 ppm to 10,000 ppm, and including 4,000 to 6,000 ppm. The sodium chlorite can be activated in a buffer having a pH that is less than or equal to 5. The sodium chlorite can be activated in a buffer having a pH that is up to about 10. The antiseptic composition can further include a surfactant. The surfactant can be a non-ionic surfactant in an amount of between 0.015% w/v to about 1.0% w/v. The non-ionic surfactant can include one or more of polyoxyethylene sorbitan monooleate, polyoxyethylene lauryl ether, or poly(ethylene glycol)-block-poly(propylene glycol)-block-poly(ethylene glycol). The composition can have antimicrobial, anti-fungal, anti-viral, and anti-parasitic activity. The antiseptic composition can be in a form of aqueous solutions, emulsions (oil-in-water or water-in-oil), lotions, creams, ointments, salves, gels, instillations, foams, powders, tinctures, and solids. The composition can be in the form of an eye drop, eye wash, eye swab, or an eye bath. The composition can be in the form of a solution, suspension, gel, swab, or bath for skin application. The tissues disinfected can include skin, eye, wound, or incision. The tissues disinfected can include skin, mucocutaneous membranes, mucous membranes, nasopharynx, lungs, eye lid, eyebrow, cheek, cornea, conjunctiva, or palpebral fornix.

In an interrelated aspect, disclosed are ophthalmically acceptable topical compositions for disinfecting ocular tissue. The composition includes sodium chlorite in an amount of about 5 ppm to about 20,000 ppm, such as 100 ppm to 10,000 ppm and including 4,000 ppm to 6,000 ppm; a surfactant in an amount of about 0.015% w/v to about 1.0% w/v; and at least one buffer. The surfactant can be polyoxyethylene sorbitan monooleate. The composition can include about 8000 ppm sodium chlorite, about 0.5% w/v polyoxyethylene sorbitan monooleate, about 0.83% w/v sodium phosphate monobasic monohydrate, about 0.17% w/v citric acid monohydrate, hydrochloric acid and/or sodium hydroxide, and water, and the composition can have a pH of about 5. The composition can include about 8000 ppm sodium chlorite, about 0.5% w/v polyoxyethylene sorbitan monooleate, about 0.25% w/v sodium phosphate monobasic monohydrate, about 0.35% w/v citric acid monohydrate, and water, and the composition can have a pH of about 4. The composition can include about 8000 ppm sodium chlorite, about 0.5% w/v polyoxyethylene lauryl ether, about 0.83% w/v sodium phosphate monobasic monohydrate, about 0.17% w/v citric acid monohydrate, hydrochloric acid and/or sodium hydroxide, and water, and the composition can have a pH of about 5. The composition can include about 8000 ppm sodium chlorite, about 0.5% w/v poly(ethylene glycol)-block-poly(propylene glycol)-block-poly(ethylene glycol), about 0.83% w/v sodium phosphate monobasic monohydrate, about 0.17% w/v citric acid monohydrate, hydrochloric acid and/or sodium hydroxide, and water, and the composition can have a pH of about 5. The at least one buffer can be a phosphate buffer, a citrate buffer, or a borate buffer. The composition can have a pH less than or equal to 7.

In an interrelated aspect, disclosed are methods for treating ocular tissue with an antiseptic composition including sodium chlorite and a buffer. Treating can include topically applying the composition to an eye of a patient. Topically applying the composition to the eye can include topically applying the composition prior to, during, and/or after development of an ocular infection, including viral conjunctivitis, bacterial conjunctivitis, bacterial keratitis, fungal keratitis, acanthamoeba keratitis, demodex infection, eyelid and eyelid margin infections, coronavirus infection, and staph marginalis. Treatment can include topical application of the composition as frequently as every 30 minutes during the duration of ocular infection or to prevent development of an infection. Treatment can include the use of the composition as prophylaxis against the development of ocular infection, including any microbial infection (viral, fungal, bacterial, parasitic, protozoan, amoeba, and so forth.

In an interrelated aspect, disclosed are methods for treating cutaneous, mucocutanous, and mucous membrane tissues with a composition including sodium chlorite and a buffer with or without a surfactant. Topically applying the composition to any tissue type including skin, can include applying the composition prior to, during, and/or after development of an infection. Treatment can include topical application of the composition as frequently as every 30 minutes during the duration of an infection or to prevent development of an infection. Treatment can be applied in an extended-release formulation, such as a gel or cream, with extended antimicrobial (as defined within).

In an interrelated aspect, disclosed is the ocular use of a composition including sodium chlorite and a surfactant. The sodium chlorite can be in an amount of about 800 ppm to about 8000 ppm. The surfactant can be in an amount of about 0.015% w/v to about 1.0% w/v. The composition can further include at least one buffer having a pH of less than or equal to 5. The composition can be topically applied to an eye tissue. The composition can be topically applied to an eye tissue prior to, during, and/or after a surgical procedure of an eye.

In an interrelated aspect, disclosed are methods for treating respiratory passages, including lung tissues with a composition including sodium chlorite and a buffer with or without a surfactant. To treat respiratory passages, the composition may be delivered via pulmonary delivery, e.g., via nebulizer or inhaler, and treatment may be delivered in discrete doses, or over a sustained period of time. In some embodiments, the nebulized form of the composition may be used to sterilize respiratory tissues.

Other features and advantages will be apparent from the following description of various embodiment, which illustrate, by way of example, the principles of the disclosed compositions and methods.

### BRIEF DESCRIPTION OF THE FIGURES

Aspects of the disclosure may be best understood from the following detailed description when read in conjunction with the accompanying drawings. Included in the drawings are the following figures:

FIG. 1 depicts a delivery device according to certain embodiments.

FIG. 2 depicts a delivery device according to certain embodiments.

FIG. 3 depicts a delivery device according to certain embodiments.

FIG. 4A-G depict delivery devices (FIG. 4A-B), seals (FIG. 4C-D), and dividers (FIG. 4E-G) according to certain embodiments.

FIG. 5 depicts a delivery device according to certain embodiments.

FIG. 6 depicts a delivery device according to certain embodiments.

FIG. 7A-B depicts a delivery device according to certain embodiments.

FIG. 8 depicts a graphical representation of Sodium Chlorite E-beam testing.

FIG. 9 provides Sodium Chlorite E-beam testing parameters and results.

FIG. 10 presents a tabular representation of the experimentally tested conditions.

FIG. 11 shows uninfected cell viability against concentration of IRX-101/vehicle.

FIG. 12 shows cell viability against concentration of Adv-5 with either RX101 or vehicle.

FIG. 13A-B depict the raw data collected for uninfected (FIG. 13A) and infected (FIG. 13B) cell viability.

FIG. 14 provides representative images of HeLa cells infected with pre-treated Human Adenovirus-5.

FIG. 15A-B depict the results of a plaque reduction assay with 1×DMEM as a diluent.

FIG. 16A-B depict the results of a plaque reduction assay with 1×PBS as a diluent.

FIG. 17A-D depict percent reduction of PFU/mL in virus exposed to compositions according to certain embodiments of the invention.

### DETAILED DESCRIPTION

There is a need for improved treatments of ocular and systemic infections, particularly viral conjunctivitis, blepharitis, dry eye syndrome, keratoconjunctivitis, sicca, bacterial conjunctivitis, bacterial keratitis, fungal keratitis, acanthamoeba keratitis, SARS-CoV-2, and the like. Chlorine dioxide is known to be a potent antimicrobial agent, although its use in clinical practice has been limited by the inherent instability in the molecule. Disclosed herein are composition of sodium chlorite and buffers that facilitate the release of stable chlorine dioxide, which can then be used to prevent and treat ocular and non-ocular infections.

Methods of treating a subject for a microbial eye condition are provided. Aspects of the methods include administering to the subject an activated sodium chlorite composition, where the compositions include sodium chlorite; and a buffer component prepared from sodium phosphate monobasic monohydrate and citric acid. Also provided are methods of inhibiting a virus associated with a tissue, such as an adenovirus or coronavirus. In addition, delivery devices for administering an activated sodium chlorite composition to a tissue are provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, aspects of the invention include treating a subject for a microbial eye condition. A variety of microbial eye conditions may be treated using compositions disclosed herein. Examples of such conditions are reviewed below.

### Adenovirus Conjunctivitis

Adenovirus is frequently implicated in viral conjunctivitis, although numerous other virus types have been noted to cause conjunctivitis. Multiple viral conjunctivitis clinical trials have failed for several reasons. The first is the fact that rapid tests to confirm the underlying etiology are not reliable. The low positive predictive value of rapid diagnostic tests for adenovirus make it very difficult to design and execute clinical trials. Specifically, it is difficult to correctly identify patients with viral conjunctivitis and then test them to show that their infection has been cleared following treatments. The second difficulty relates to the sporadic nature of viral conjunctivitis outbreaks, making it difficult to set up and run clinical trials. Finally, viruses are notoriously hard to treat, and anti-viral medications have not been successful to date.

There are more than 50 serotypes of adenovirus in subgroups A-F. Symptoms can vary from mild ocular injection and follicular conjunctivitis to severe infection involving both the conjunctiva and cornea (keratoconjunctivitis). Epidemic keratoconjunctivitis (EKC) can result in severe ocular symptoms, with the presence of pseudomembranes, subepithelial corneal infiltrates and corneal erosions. Pharyngoconjunctival fever describes adenoviral conjunctivitis with systemic symptoms including fever, sore throat, and headache. Viral conjunctivitis is extremely contagious and can be transmitted via respiratory or ocular secretions, contaminated objects (mascara brushes, eye drop bottles, door handles) and even contaminated swimming pools.

Previous studies have investigated a variety of therapies to treat viral conjunctivitis. These have included studies of various antiseptics, including povidone-iodine (PI). While there is anecdotal evidence that povidone-iodine may help treat EKC, this medication has a very significant limitation—significant ocular toxicity. Povidone-iodine causes severe pain upon instillation into the eye and results in corneal epithelial toxicity. To use this as treatment for patients requires patients to come into the doctors' office for application of topical lidocaine before application of PI. This is not feasible for widespread use, particularly given the high percentage of pediatric patients with infections. The compositions described herein have the advantage of causing minimal to no ocular toxicity and have been well tolerated following topical application.

Compositions disclosed herein can be used to effectively treat viral conjunctivitis, including adenoviral conjunctivitis and EKC. Due to the tolerability of the solutions disclosed herein, they are suitable for patient administration, including dosing as frequent as an eye rinse or every 30-minute dosing. Compositions disclosed herein can include combination medications, such as purified sodium chlorite and a buffer with or without a surfactant and a steroid such as dexamethasone or prednisolone co-formulated with either the buffer or purified sodium chlorite. The compositions can be delivered as multiple aseptic single-use vials or blow fill seal containers that lend themselves to single dosing followed by discard of residual drops. The compositions described herein can be provided in 2 containers and mixed shortly before use, with instructions to use within 1 hour of mixing.

### Coronavirus Conjunctivitis

In early 2020, reports began to emerge of ocular involvement in patients with SARS-CoV-2. In late March, a report published in JAMA Ophthalmology provided data showing that 31.6% of patients in their cohort of 38 subjects manifested signs of ocular infection.¹ These signs included conjunctival hyperemia, chemosis, epiphora, and increased secretions. In addition, 2 subjects had SARS-CoV-2 isolated from their tears, and the authors concluded that SARS-CoV-2 can be transmitted via the ocular route. With regard to COVID-19, unprotected ocular exposure was thought to be responsible for infections that occurred in the Wuhan Fever Clinic in January 2020.²⁻⁴ Colavita and colleagues reported findings on the first patient with COVID-19 in Italy.⁴ She presented with conjunctivitis, and ocular swabs were obtained almost daily for over 4 weeks demonstrating replicating SARS-CoV-2 for weeks. Ocular samples were inoculated in Vero cells, with cytopathic effect noted 5 days post-inoculation. Interestingly, ocular tear samples continued to showcase replicating virus longer than nasopharyngeal swabs, and viral counts were again detectable on day 27, about a week after ocular symptoms resolved, suggesting sustained conjunctival replication.

The importance of these studies is clear, demonstrating that SARS-CoV-2 can both infect the eyes, and be present in ocular secretions even in the absence of symptoms. This presents another form of disease transmission, and can lead to more widespread dissemination of infection. This is particularly important in specialties like ophthalmology and optometry, where patient care involves close patient contact and examination of the eyes. In fact, early reports suggest that ophthalmologists, along with emergency room and critical care physicians, are among the most likely health care providers to develop infection. While reports highlight the importance of ophthalmologists wearing eye protection,⁵ this equipment is not always available, and in many cases, does not work with ophthalmic examination equipment. For example, face shields prevent the use of an indirect ophthalmoscope, a crucial instrument for examining the anterior retina.

The compositions described herein are well tolerated following ocular application, and as such, can be used to effectively prevent ocular coronavirus infection or to treat active infection. They can be used by health care providers prior to or immediately following interactions with others including patients, and can be used by people immediately before or after interacting with others. For example, the compositions can be activated and applied to the eyes immediately before a visit to the doctor to effectively sterilize the ocular surface prior to an examination. In another embodiment, the composition can be used by patients with known coronavirus infection to maintain ocular surface sterility and reduce the likelihood of transmission of the disease following exposure of ocular secretions. In another embodiment, the compositions described herein can be used to treat active ocular coronavirus infection of the eye, including SARS-CoV-2 conjunctivitis. Treatment may include a lavage of the ocular surface, or drops applied as frequently as every 5 minutes or as infrequently as a single use.

### Blepharitis Treatment

Blepharitis is one of the most common ophthalmic disorders, affecting millions of patients in the United States alone. Blepharitis is a multifactorial disorder, and infectious etiologies have been implicated in the disease. Demodex infestation has been found in up to 30% of patients with chronic blepharitis. Phthirus pubis can also cause blepharitis. Staphylococcal species blepharitis is felt to be caused by either direct irritation of the eye and lids from bacterial toxins or enhanced cell-mediated immunity to S. aureus and related species. Bacterial infestation of the eyelid margin has been implicated in numerous studies. Herpes simplex virus and molluscum contagiuosum can also result in blepharitis. Moraxella can also lead to a chronic angular blepharoconjunctivitis. Streptococcus species, HSV, and VZV can also result in a dermatoblepharitis.

The compositions disclosed herein can be used to effectively treat bacterial or other antimicrobial contributors to blepharitis. The compositions described herein can be applied via any format described herein, including, but not limited to drops, ointments, swabs, lid scrubs, salves, and the like. Staph marginal keratitis is also caused by bacterial infection, and the compositions described herein can be used as an effective treatment. Phylyctenules can also have an infectious nidus and be treated by compositions described herein. The compositions can be formulated for use as an over the counter eye cleansing solution or a prescription medication to reduce antimicrobial and parasitic infestation that contributes to dry eye and blepharitis.

### Bacterial Conjunctivitis

Bacterial Conjunctivitis tends to result in a more virulent clinical picture than non-EKC viral conjunctivitis. Species frequently implicated include Chlamydia trachomatis, Staphylococcus aureus, Streptococcus spp, Neisseria gonorrhoeae, Haemophilus influenzae, and Moraxella spp.

### Neonatal Conjunctivitis

Neonatal conjunctivitis frequently manifests with severe conjunctivitis and can also lead to pneumonia. Bacterial causes include Chlamydia trachomatis (most common), Neisseria gonorrhea, S. aureus, Pseudomonas aeruginosa, Streptococcus spp, Klebsiella, Proteus, Enterobacter, Serratia, and Eike Nella corroden. Viral causes include herpes simplex virus. The compositions described herein have broad antimicrobial properties with an excellent safety profile, and can be used prophylactically for infants immediately following birth to reduce the risk of neonatal conjunctival infection. The compositions described herein can also be used on an ongoing basis to treat neonatal conjunctivitis.

### Bacterial Keratitis

Bacterial keratitis, or corneal ulcers, are a frequent cause of ocular morbidity. These can range from transient infections, to fulminant disease that melts the cornea and results in loss of vision and loss of the eye. Many different microbes can cause infection, including, but not limited to Neisseria, Corynebacterium, Shigella, Haemophilus aegyptus, Listeria monocytogenes, Staphylococcus, Streptococcus, enteric gram-negative bacilli and gram-positive bacilli, and pseudomonas. Development of multi-drug resistant microbes poses a particular treatment in the treatment of these infections, and severe infections frequently require treatment with compounded anti-biotics that are not readily available, are expensive, and result in significant delays in treatment. A broad-spectrum treatment for bacterial keratitis would be useful in part because of the lack of development of antibiotic or anti-infective resistance of microbes.

Compositions described herein can be applied to the ocular surface, eyelids, eyelid margins, and eyelashes to successfully penetrate biofilm and scurf and treat underlying bacterial colonization, enabling improved function of healthy eyelid tissues and restoring a more natural tear film composition. Any dosage form described herein can be utilized for this indication, including eye drops, salves, ointments, lid scrubs, cotton-tipped applicators, and the like.

### Acanthamoeba Keratitis (AK)

AK is a rare, extremely virulent parasitic infection that frequently results in permanent vision loss for patients. It is frequently seen in contact lens wearers and is difficult to diagnose and even more difficult to treat. Acanthamoeba exists as both cysts and trophozoites and is a free-living amoeba that can be present in pools, hot tubs, tap water, shower water, and contact lens solutions. AK is characterized by pain that is out of proportion to the clinical examination. There are no FDA approved treatments, and a wide variety of therapies are used in an attempt to slow progression of disease. Due in large part to the lack of effective treatments, patients frequently progress to corneal melt and require corneal transplantation. In some cases, the infection results in secondary endophthalmitis and loss of the eye (enucleation). Compositions described herein can be used to treat AK.

### Fungal Keratitis

Fungal keratitis is an ocular infection that can have devastating effects on vision. Fungal ocular infections are caused by molds (both septate and nonseptate fungi molds) and yeasts like Candida and Cryptococcus. Implicated species include, but are not limited to Fusarium spp, Aspergillus spp, and Candida albicans. Current treatments are limited by cost, availability, and narrow spectrum of treatment. Developing a broad-spectrum treatment for fungal keratitis has considerable clinical value for patients. Compositions described herein can be used to treat fungal keratitis, and have the benefit of being broad spectrum with low ocular toxicity.

### Compositions

Described herein are compositions containing sodium chlorite activated in a buffer, i.e., activated sodium chlorite compositions. The antiseptic compositions provide antimicrobial activity, in particular to eye tissues, with less ocular irritation and toxicity compared to povidone-iodine ophthalmic solutions. In some implementations, the compositions containing activated sodium chlorite include a surfactant and are up to 50,000 times more effective than povidone-iodine (Betadine®) as a rapid-onset anti-microbial agent without the ocular irritation and toxicity associated with povidone-iodine ophthalmic solution. In some implementations, the sodium chlorite is formulated at concentrations ≥10 ppm sodium chlorite, activated with buffers at pH≤8, and include non-ionic surfactants (e.g. polyoxyethylene sorbitan monooleate (polysorbate-80 or PS-80), polyoxyethylene lauryl ether (Brij-35), or Pluronic F-127) at concentrations ranging from 0.05% to 1.0%. The anti-microbial efficacy of the sodium chlorite composition having PS-80 showed an unexpected efficacy over sodium chlorite compositions having other non-ionic surfactants.

“Antiseptic,” as used herein, may be used to refer to a substance that can be used on living tissues for its antimicrobial activity. “Antimicrobial,” as used herein, may be used to refer to a substance that kills or inhibits reproduction of pathogens, including but not limited to bacteria, viruses, fungi, protozoans, parasites, and so forth.

“Infection,” as used herein, may be used to refer to an invasion of an organism's body tissues by a pathogen, any multiplication of the invading pathogen in a bodily tissue, and/or any toxins or reactions (including immunological reactions) caused by such invasion. Pathogens may include bacteria, viruses, fungi, protozoans, parasites, and so forth. Infections may occur in infection sites such as eyes; ears; nasal passages; the buccal or tracheal passages or lungs; skin sites including hands, fingers, feet, and toes; genitourinary passages including the vagina and urethra; the bladder; the prostate, cuts, abrasions, lacerations, fistulae, pressure sores, ulcers, cellulitis, boils, impetigo, athletes foot, warts, and the like.

As used herein, “sodium chlorite” refers to “stabilized chlorine dioxide,” commercially available as Purite® (AGN-238749-Z), which is an aqueous solution of sodium chlorite (NaClO₂). Various implementations containing stabilized chlorine dioxide contemplated herein include all forms of sodium chlorite salts or solutions, as well as other chlorite salts and/or chlorite solutions not containing sodium (for example but without limitation, lithium, potassium, calcium, magnesium, zinc). In some instances, the sodium chlorite is substantially free of heavy metals, e.g., having 2.0 ppm or less heavy metals. In some instances, the sodium chlorite has little if any chlorate ion, such as 400 ppm or less, e.g., 300 ppm or less, including 200 ppm or less chlorate ion.

As used herein, “sodium chlorite” or “purified sodium chlorite” can comprise less than 2.0 ppm heavy metals, less than 400 ppm chlorate ion, 2.10-2.30% w/v titratable ClO₂, pH between 8.0 and 9.0, and a spectral analysis less than 0.10 A.U. at 400 nm blanked against D.I. water. In some embodiments, heavy metals can comprise less than 100 ppm, such as 0.01 ppm to 200 ppm. In some embodiments, chlorate ion can comprise less than 2000 ppm, such as 1 ppm to 200 ppm. In some embodiments the titratable ClO₂ ranges from 0.01% to 20% w/v, such as 0.1% w/v to 4.5% w/v. In some embodiments, the pH ranges from 5.0 to 10.0, such as 7.0 to 8.5.

Chlorine dioxide (ClO₂) can be generated from sodium chlorite (NaCl₂) upon activation with a buffer. The generation of chlorine dioxide from sodium chlorite (NaClO₂) can be represented by the equation:

5NaClO₂+5H⁺→[HClO₂]→4ClO₂+2H₂O+HCl+5Na⁺.

The sodium chlorite can be activated with a buffer having a pH less than or equal to pH 5, such as a pH 2, pH 3, pH 4, or pH 5. Sodium chlorite in the presence of a pH 5.0 activating buffer provides approximately 0.1% chlorine dioxide. Sodium chlorite in the presence of a pH 4.0 activating buffer provides approximately 1.0% chlorine dioxide (or 10×pH 5.0). The sodium chlorite concentrations may be described herein in ppm (parts per million). The source of sodium chlorite can be Purite®, which is typically provided as a 2.0% stock solution, where the percentage refers to the percent of potential chlorine dioxide generated from the sodium chlorite in the stock solution. Table 1 below provides an explanation for the conversion of % Purite®, where % w/v or ppm of Purite® represents the potential chlorine dioxide concentration achieved upon activation of the sodium chlorite contained in the Purite®. The % w/v (ppm) of sodium chlorite assumes a stoichiometric conversion (80% yield) of sodium chlorite into chlorine dioxide.

TABLE 1

| % Purite ® | | | | | |
|---|---|---|---|---|---|
| (potential chlorine dioxide) | | mM Purite ® | % NaClO₂* | | mM NaClO₂ |
| 2.0 | (20,000 ppm) | 296 | 3.35 | (33,500 ppm) | 370 |
| 1.0 | (10,000 ppm) | 148 | 1.68 | (16,800 ppm) | 185 |
| 0.5 | (5,000 ppm) | 74 | 0.84 | (8,400 ppm) | 93 |
| 0.1 | (1,000 ppm) | 14.8 | 0.168 | (1,680 ppm) | 18.35 |
| 0.05 | (500 ppm) | 7.4 | 0.084 | (840 ppm) | 9.3 |
| 0.01 | (100 ppm) | 1.48 | 0.0168 | (168 ppm) | 1.85 |
| 0.005 | (50 ppm) | 0.74 | 0.0084 | (84 ppm) | 0.93 |
| 0.0001 | (1 ppm) | 0.0148 | 0.000168 | (1.68 ppm) | 0.0186 |

*Assumes a stoichiometric conversion (80% yield) of sodium chlorite into chlorine dioxide.

In some implementations, the composition contains at least about 0.08% w/v sodium chlorite or about 800 ppm sodium chlorite up to about 2.2% w/v sodium chlorite or about 22,000 ppm sodium chlorite). In other implementations, the sodium chlorite concentrations in the compositions include 0.08% w/v, 0.085% w/v, 0.09% w/v, 0.095% w/v, 0.10% w/v, 0.15% w/v, 0.30% w/v, 0.35% w/v, 0.40% w/v, 0.45% w/v, 0.50% w/v, 0.55% w/v, 0.60% w/v, 0.65% w/v, 0.70% w/v, 0.75% w/v, 0.80% w/v, and 0.85% w/v, and may all be used in conjunction with the implementations described herein.

In some implementations, the composition contains at least about 0.005% w/v Purite® or about 50 ppm Purite® up to about 0.5% w/v Purite® or about 5000 ppm Purite®), wherein the % w/v or ppm represents potential chlorine dioxide upon activation of the sodium chlorite in the Purite®. In other implementations, the Purite® concentrations in the composition include 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.10% w/v, 0.15% w/v, 0.20% w/v, 0.25% w/v, 0.30% w/v, 0.35% w/v, 0.40% w/v, 0.45% w/v, 0.50% w/v, and may all be used in conjunction with the implementations described herein.

In some implementations, the sodium chlorite is activated by one or more activating buffers. Example buffers considered herein include, but are not limited to, acetate buffers, citrate buffers, phosphate buffers, borate buffers, lactate buffers, NaOH/trolamine buffers, or a combination thereof, such as phosphate and citrate or borate and citrate. In some implementations the buffer is sodium phosphate monobasic monohydrate and citric acid monohydrate (see Table 2). In other implementations, the buffer is sodium borate, decahydrate. Acids or bases, such as HCl and NaOH, may be used to adjust the pH as needed. The activating buffer can have a pH 2, pH 3, pH 4, pH 5, pH 6, or pH 7. In other implementations, the pH of the activating buffer can be less than or equal to pH 5. In other implementation, the pH of the activating buffer can be up to about pH 7.6. The amount of buffer used may vary. In some embodiments, the buffer may have a concentration in a range of about 1 nM to about 100 mM.

TABLE 2
Buffer composition for sodium chlorite activation

| | Buffer | Buffer | Buffer | Buffer |
|---|---|---|---|---|
| Buffer | for pH 2 | for pH 3 | for pH 4 | for pH 5 |
| Ingredients | % w/v | | | |
| Sodium Phosphate | 0.15 | 0.15 | 0.25 | 0.83 |
| Monobasic | | | | |
| Monohydrate | | | | |
| Citric Acid | 1.0 | 1.0 | 0.35 | 0.17 |
| Monohydrate | | | | |
| Hydrochloric acid | 6 | 0 | 0 | 0 |
| 1N | | | | |
| Sodium Hydroxide | 0 | 0 | 0 | 0.92 |
| 1N | | | | |

The composition can further include one or more co-solubilizers such as a surfactant. The surfactant may vary, and may include any compound that is surface active or can form micelles. A surfactant may be used for assisting in dissolving an excipient or an active agent, dispersing a solid or liquid in a composition, enhancing wetting, modifying drop size, stabilizing an emulsion, or a number of other purposes. Examples of surfactants may include, but are not limited to, surfactants of the following classes: alcohols, for example polyvinyl alcohol; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal & veg.); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants such as Soluplus® (from BASF); propoxylated & ethoxylated fatty acids, alcohols, or alkyl phenols; protein-based surfactants; sarcosine derivatives; sorbitan derivatives; sucrose and glucose esters and derivatives; and saponins. In some embodiments, the surfactant may include polyethylene glycol (15)-hydroxystearate (CAS Number 70142-34-6, available as SOLUTOL HS 15® from BASF), a polyoxyethylene-polyoxypropylene block copolymer (CAS No. 9003-11-6, available as PLURONIC® F-68 from BASF), polyoxyethylene 40 stearate (POE40 stearate), polysorbate 80 or polyoxyethylene (80) sorbitan monooleate (CAS No. 9005-65-6), sorbitan monostearate (CAS No. 1338-41-6, available as SPAN™ 60 from Croda International PLC), or polyoxyethyleneglyceroltriricinoleate 35 (CAS No. 61791-12-6, available as CREMOPHOR EL® from BASF), ethoxylated castor oil, such as Cremophor EL (CAS Number 61791-12-6). Suitable co-solubilizers include, but are not limited to, povidone, and acrylates (e.g. PEMULEN®).

In some implementations, the surfactant is a non-ionic surfactant that can include polyoxyethylene sorbitan monooleate (Polysorbate-80) represented by CAS No. 9005-65-6, such as Tween® 80, available from Sigma-Aldrich. In some implementations, the non-ionic surfactant includes polyoxyethylene lauryl ether represented by CAS No. 9002-92-0, such as Brij® 35, available from Sigma-Aldrich. In some implementations, the non-ionic surfactant polyol includes poly(ethylene glycol)-block-poly(propylene glycol)-block-poly(ethylene glycol) represented by CAS No. 9003-11-6, such as Pluronic™ F-127, available from BASF SE. Other non-ionic surfactants are considered herein including, but not limited to ethoxylates, fatty alcohol ethoxylates, alkylphenol ethoxylates, fatty acid ethoxylates, special ethoxylated fatty esters and oils, ethoxylated amines and/or fatty acid amides, terminally blocked ethoxylates, fatty acid esters of polyhydroxy compounds, fatty acid esters of glycerol, fatty acid esters of sorbitol, Tweens, fatty acid esters of sucrose, alkyl polyglucosides, amine oxides, sulfoxides, phosphine oxides.

It should be appreciated that the surfactant incorporated in the compositions is not limited by class and that various classes of surfactants can be incorporated including, but not limited to anionic, cationic, zwitterionic, and nonionic surfactants. It should also be appreciated combinations of surfactants can be included.

The amount of surfactant may vary. In some implementations, the surfactant can be used at a concentration from about 0.05% w/v to about 5.0% w/v, 0.05% w/v to about 0.5% w/vv. Some preferred concentrations of the surfactant include 0.04% w/v, 0.045% w/v, 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.10% w/v, 0.15% w/v, 0.20% w/v, 0.25% w/v, 0.30% w/v, 0.35% w/v, 0.40% w/v, 0.45% w/v, 0.50% w/v, 0.55% w/v, 0.60% w/v, 0.65% w/v, 0.70% w/v, 0.75% w/v, 0.80% w/v, 0.85% w/v, 0.90% w/v, 0.95% w/v, 1.0% w/v, 1.5% w/v, 2.0% w/v, 2.5% w/v, 3.0% w/v, 3.5% w/v, 4.0% w/v, 4.5% w/v, and 5.0% w/v and may all be used in conjunction with the implementations described herein.

In some implementations, the composition can include ≥800 ppm sodium chlorite activated in an activating buffer having a pH≤5, and added polysorbate 80 at a concentration between about 0.25% up to about 0.5%. Table 3 provides various compositions of sodium chlorite containing polysorbate 80 (PS80).

TABLE 3

| Sodium Chlorite | pH of activating | PS80 |
|---|---|---|
| (ppm) | buffer | (% w/v) |
| 800 | 4 | 0.25 |
| 1000 | 4 | 0.25 |
| 2000 | 4 | 0.25 |
| 3000 | 4 | 0.25 |
| 4000 | 4 | 0.25 |
| 5000 | 4 | 0.25 |
| 6000 | 4 | 0.25 |
| 7000 | 4 | 0.25 |
| 8000 | 4 | 0.25 |
| 800 | 5 | 0.25 |
| 1000 | 5 | 0.25 |
| 2000 | 5 | 0.25 |
| 3000 | 5 | 0.25 |
| 4000 | 5 | 0.25 |
| 5000 | 5 | 0.25 |
| 6000 | 5 | 0.25 |
| 7000 | 5 | 0.25 |
| 8000 | 5 | 0.25 |
| 800 | 4 | 0.5 |
| 1000 | 4 | 0.5 |
| 2000 | 4 | 0.5 |
| 3000 | 4 | 0.5 |
| 4000 | 4 | 0.5 |
| 5000 | 4 | 0.5 |
| 6000 | 4 | 0.5 |
| 7000 | 4 | 0.5 |
| 8000 | 4 | 0.5 |
| 800 | 5 | 0.5 |
| 1000 | 5 | 0.5 |
| 2000 | 5 | 0.5 |
| 3000 | 5 | 0.5 |
| 4000 | 5 | 0.5 |
| 5000 | 5 | 0.5 |
| 6000 | 5 | 0.5 |
| 7000 | 5 | 0.5 |
| 8000 | 5 | 0.5 |

In some implementations, the activated sodium chlorite compositions may be prepared in the form of a solution, for example a solution using a physiological saline solution as a major vehicle. Solutions may be maintained at a comfortable pH with an appropriate buffer system. The formulations may also contain conventional, pharmaceutically acceptable preservatives, stabilizers, and surfactants. In some implementations, the composition is formulated as an ophthalmically acceptable liquid or solution.

Certain liquid compositions may include an osmolality agent. The osmolality agent may vary, and may include any compound or substance useful for adjusting the osmolality of a liquid. Examples include, but are not limited to, salts, particularly sodium chloride or potassium chloride, organic compounds such as propylene glycol, mannitol, or glycerin, or any other suitable osmolality adjustor. In some embodiments, an osmolality agent may comprise propylene glycol, glycerin, mannitol, sodium chloride, or a combination thereof. The amount of osmolality agent may vary depending upon whether an isotonic, hypertonic, or hypotonic liquid is desired. In some embodiments, the amount of an osmolality agent such as those listed above may be at least about 0.0001% w/w up to about 1% w/w, about 2% w/w, or about 5% w/w.

As described above, the sodium chlorite can be activated in a buffer generating chlorine dioxide prior to formulation of the final composition to be applied to the eye, skin, or other target treatment area. In some implementations, the activating buffer can be a citrate or phosphate buffer considered suitable for lower pH solutions. In other implementations, the activating buffer can be a borate buffer considered suitable for higher pH solutions (e.g. in the pH 7 range). The citrate and phosphate activating buffers can be sufficient to achieve desired final isotonicity of the final ophthalmic solution from these relatively low pH solutions. The borate buffers, in contrast, may include additional osmolality agents, such as glycerol, to achieve desired final isotonicity.

In some embodiments, an additional co-solubilizer may comprise sorbitan monostearate, a polyoxyethylene-polyoxypropylene block copolymer, polyoxyethyleneglyceroltriricinoleate 35, a cyclodextrin, or a combination thereof. Certain compositions may include an antioxidant. The antioxidant may vary, and may include any compound or substance that is useful in reducing oxidation of any compound present in the composition. Examples include, but are not limited to, citrate, L-carnosine, oleic acid, and zinc. Certain compositions may include a chelating agent. The chelating agent may vary, and may include any compound or substance that is capable of chelating a metal. A useful chelating agent is edetate disodium, although other chelating agents may also be used in place or in conjunction with it.

In some embodiments, compositions may include one or more viscosity enhancers. For example, a viscosity enhancer may comprise an acrylic acid or acrylate polymer, either cross-linked or non-cross-linked such as polycarbophil, for example CARBOPOL® (B.F. Goodrich, Cleveland, Ohio) and CARBOPOL 980®. These polymers may dissolve in water and may form a clear or slightly hazy gel upon neutralization with a base such as sodium hydroxide, potassium hydroxide, triethanolamine, or other amine bases. Other commercially available thickeners may include HYPAN® (Kingston Technologies, Dayton, N.J.), NATROSOL® (Aqualon, Wilmington, Del.), KLUCEL® (Aqualon, Wilmington, Del.), or STABILEZE® (ISP Technologies, Wayne, N.J.). KLUCEL® is a cellulose polymer that may be dispersed in water and may form a uniform gel upon complete hydration. Other useful gelling polymers may include carboxymethyl cellulose, hydroxyethyl cellulose, hydroxypropylcellulose, cellulose gum, MVA/MA copolymers, MVE/MA decadiene crosspolymer, PVM/MA copolymer, etc.

In some implementations, the composition takes the form of an aqueous solution configured to be applied as a drop, wash, swab, or bath. Other suitable forms are emulsions (oil-in-water or water-in-oil), lotions, creams, ointments, salves, gels, instillations, foams, powders, tinctures, solids, and so forth. The composition is configured to be administered topically to a body surface, which may include sites such as the eye, skin, mucous membranes, incision site, wound location, or other treatment site.

In some implementations, the composition can be provided as part of a kit where the final composition is mixed together by an end user prior to use. For example, a first formulated part (e.g. a buffer solution with or without one or more other excipients such as a surfactant mixed to a desired concentration, tonicity agent, etc.) can be provided in a first container and a second formulated part (e.g. a sodium chlorite stock solution) provided in a second container. The two formulated parts can be mixed together to form the final composition (e.g. as a 1:1, 1:2, 1:3, 2:3, 1:5, 1:4, or other mixture). The two parts can be mixed together before being dispensed.

The compositions described herein can be filled aseptically or in a clean environment. If they are not filled in an aseptic, ISO 5 standard room or similar, terminal sterilization can be undertaken to verify an appropriately low bioburden. Any method of terminal sterilization can be used, including autoclaving, gamma radiation, ethylene oxide sterilization, electron beam sterilization and the like. To facilitate sodium chlorate stability during terminal sterilization, the product can be cooled to temperatures ranging from 20 C to −80 C, and can undergo terminal sterilization by e-beam, gamma radiation, or other means while frozen. Radiation doses from 1 to 100 kGy can be used to sterilize compositions described herein.

The compositions described herein can have enhanced bacterial kill efficacy compared to povidone-iodine (i.e. Betadine®) without the associated ocular toxicity. Thus, the compositions described herein can be used similarly to how povidone-iodine is currently used with greater efficacy in anti-microbial kill and with little to no toxicity to the ocular tissue. The compositions described herein can be used prophylactically, prior to exposure to a pathogen capable of causing an infection or prior to the establishment of an infection. In some implementations, the composition can be administered to a treatment site as a single, one-time application sufficient to disinfect and prepare the treatment site for a surgical procedure. In another embodiment, the composition can be applied 1 week to 30 minutes before a procedure or surgery, such as 1 to 8 hours before, and again immediately before a procedure or surgery. The compositions described herein are useful as an antimicrobial preparation for all ocular procedures, for example, invasive procedures including intraocular injections, including but not limited to intravitreal, intracorneal, scleral, sub-Tenon's, intracameral, subretinal, minimally invasive glaucoma procedures, suture removal, or sub-conjunctival injections, as well as all ocular surgical procedures, including cataract and lens surgeries, trabeculectomy, vitrectomy, scleral buckle, glaucoma tube shunt surgery, pterygium removal, corneal transplants, eyelid and orbital surgeries, reconstructive and cosmetic facial surgery, ocular oncology surgeries, iris surgery, choroidal and subretinal surgery, strabismus surgery, ocular trauma surgery etc. Thus, the compositions can be formulated as an eye drop, eye wash, eye swab, or an eye bath for use on eye lids, eyebrow, cheek, cornea, conjunctiva, palpebral fornices, etc.

Although the compositions are described herein as configured for ocular applications, they should not be limited as such. The compositions described herein can be applied topically to a variety of body surfaces, including the eye, ear, skin, nails, mucocutaneous membranes, or mucous membranes. The compositions described herein can be used as pre-surgical site sterilization prep for all surgical procedures, including all non-ocular surgical procedures that require a sterile surgical site preparation. The compositions can be applied as a solution, salve, ointment, or application stick, and are designed to be used prior to or immediately after drying, with residual effect for up to 24 hours post application. The compositions can be useful in non-ocular skin applications, including applications where a biofilm can present bacteria prior to a surgery where a robust bacterial kill is desired, for example, implant surgeries characterized by creating pockets in tissues that are washed out with antiseptics prior to positioning an implant (e.g. breast implant surgery, orthopedic surgery with implants, gynecologic surgical implant surgery, neurosurgical or spine surgery with implants).

It will be noted that while some implementations described herein may be suitable as a prophylactic agent, the implementations are not limited as such. The compositions are specifically envisioned as a treatment of active ocular and non-ocular infections, including active skin infections like cellulitis, as described elsewhere in this application. The compositions can be used for the treatment (e.g. cleansing) of an existing infected or non-infected wound or surgical incision site (e.g. ocular or non-ocular site). The compositions can be administered at least once a day to a treatment site. In some embodiments, the compositions may be delivered on a continuous basis, for example as a nebulized dosage form. In other embodiments, the composition may be applied, 1, 2, 3, 4, 5, 6, 7, 8, 9, or 10 times a day to the treatment site. In other embodiments, the compositions may be applied up to 100 times a day. In another embodiment, the composition may be applied every 15 minutes around the clock, or at an interval between every 15 minutes and every 24 hours. The implementations described herein may also be usable in a veterinary context, and not only for the treatment of humans.

Tables 4-7 below lists various examples of compositions considered herein that are ophthalmically acceptable topical antiseptics for ocular tissues and also acceptable for cutaneous tissues.

TABLE 4

| Part A Composition (2% Purite stock) | | |
|---|---|---|
| Sodium chlorite | 36.85 | mg/mL |
| Sodium chloride | 2.75 | mg/mL |
| Sodium hydrogen carbonate | 2 | mg/mL |
| Sodium formate | 0.94 | mg/mL |
| Methanol | 0.5 | mg/mL |
| Sodium chlorate | 0.16 | mg/mL |
| Water | 956 | mg/mL |
| Part B Composition (Buffer Solution) | | |
| Polysorbate 80 | 0.50% | w/v |
| Sodium phosphate monobasic monohydrate | 0.83% | w/v |
| Citric acid monohydrate | 0.17% | w/v |

| | |
|---|---|
| Sodium hydroxide 1N | pH adjust |
| Final pH after A:B reconstitution | 5 |

TABLE 5

| Part A Composition (2% Purite stock) | | |
|---|---|---|
| Sodium chlorite | 36.85 | mg/mL |
| Sodium chloride | 2.75 | mg/mL |
| Sodium hydrogen carbonate | 2 | mg/mL |
| Sodium formate | 0.94 | mg/mL |
| Methanol | 0.5 | mg/mL |
| Sodium chlorate | 0.16 | mg/mL |
| Water | 956 | mg/mL |
| Part B Composition (Buffer Solution) | | |
| Polysorbate 80 | 0.50% | w/v |
| Sodium phosphate monobasic monohydrate | 0.25% | w/v |
| Citric acid monohydrate | 0.35% | w/v |

| | |
|---|---|
| Final pH after A:B reconstitution | 4 |

TABLE 6

| Part A Composition (2% Purite stock) | | |
|---|---|---|
| Sodium chlorite | 36.85 | mg/mL |
| Sodium chloride | 2.75 | mg/mL |
| Sodium hydrogen carbonate | 2 | mg/mL |
| Sodium formate | 0.94 | mg/mL |
| Methanol | 0.5 | mg/mL |
| Sodium chlorate | 0.16 | mg/mL |
| Water | 956 | mg/mL |
| Part B Composition (Buffer Solution) | | |
| Polysorbate 80 | 0.50% | w/v |
| Sodium phosphate monobasic monohydrate | 0.15% | w/v |
| Citric acid monohydrate | 1.0% | w/v |

| | |
|---|---|
| Final pH after A:B reconstitution | 3 |

TABLE 7

| Part A Composition (2% Purite ® stock) | | |
|---|---|---|
| Sodium chlorite | 36.85 | mg/mL |
| Sodium chloride | 2.75 | mg/mL |
| Sodium hydrogen carbonate | 2 | mg/mL |
| Sodium formate | 0.94 | mg/mL |
| Methanol | 0.5 | mg/mL |
| Sodium chlorate | 0.16 | mg/mL |
| Water | 956 | mg/mL |
| Part B Composition (Buffer Solution) | | |
| Polysorbate 80 | 0.5% | w/v |
| Sodium phosphate monobasic monohydrate | 0.15% | w/v |
| Citric acid monohydrate | 1.0% | w/v |

| | |
|---|---|
| Hydrochloric acid 1N | pH adjust |
| Final pH after A:B reconstitution | 2 |

Table 8 below lists various examples of compositions considered herein that are ophthalmically acceptable topical antiseptics for ocular tissues.

TABLE 8

| Composition 1 | | |
|---|---|---|
| Sodium chlorite | 8000 | ppm |
| Polysorbate 80 | 0.015% | w/v |
| Sodium phosphate monobasic monohydrate | 0.25% | w/v |
| Citric acid monohydrate | 0.35% | w/v |
| pH | 4 | |
| Composition 2 | | |
| Sodium chlorite | 8000 | ppm |
| Polysorbate 80 | 0.25% | w/v |
| Sodium phosphate monobasic monohydrate | 0.25% | w/v |
| Citric acid monohydrate | 0.35% | w/v |
| pH | 4 | |
| Composition 3 | | |
| Sodium chlorite | 8000 | ppm |
| Polysorbate 80 | 0.50% | w/v |
| Sodium phosphate monobasic monohydrate | 0.25% | w/v |
| Citric acid monohydrate | 0.35% | w/v |
| pH | 4 | |
| Composition 4 | | |
| Sodium chlorite | 800 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 5 | | |
| Sodium chlorite | 1600 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 6 | | |
| Sodium chlorite | 3200 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 7 | | |
| Sodium chlorite | 4800 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 8 | | |
| Sodium chlorite | 6400 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 9 | | |
| Sodium chlorite | 8000 | ppm |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 10 | | |
| Sodium chlorite | 8000 | ppm |
| Sodium phosphate monobasic monohydrate | 0.25% | w/v |
| Citric acid monohydrate | 0.35% | w/v |
| pH | 4 | |
| Composition 11 | | |
| Sodium chlorite | 8000 | ppm |
| Sodium phosphate monobasic monohydrate | 0.15% | w/v |
| Citric acid monohydrate | 1.0% | w/v |
| pH | 3 | |
| Composition 12 | | |
| Sodium chlorite | 8000 | ppm |
| Sodium phosphate monobasic monohydrate | 0.15% | w/v |
| Citric acid monohydrate | 1.0% | w/v |
| Hydrochloric acid 1N | pH adjust | |
| pH | 2 | |
| Composition 13 | | |
| Sodium chlorite | 8000 | ppm |
| Polysorbate 80 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 14 | | |
| Sodium chlorite | 8000 | ppm |
| Brij 35 | 0.015% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 15 | | |
| Sodium chlorite | 8000 | ppm |
| Brij 35 | 0.25% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 16 | | |
| Sodium chlorite | 8000 | ppm |
| Brij 35 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 17 | | |
| Sodium chlorite | 8000 | ppm |
| PF127 | 0.015% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 18 | | |
| Sodium chlorite | 8000 | ppm |
| PF127 | 0.25% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 19 | | |
| Sodium chlorite | 8000 | ppm |
| PF127 | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 20 | | |
| Sodium chlorite | 8000 | ppm |
| Saponin | 0.015% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 21 | | |
| Sodium chlorite | 8000 | ppm |
| Saponin | 0.25% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 22 | | |
| Sodium chlorite | 8000 | ppm |
| Saponin | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 23 | | |
| Sodium chlorite | 8000 | ppm |
| CMC | 0.015% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 24 | | |
| Sodium chlorite | 8000 | ppm |
| CMC | 0.25% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 25 | | |
| Sodium chlorite | 8000 | ppm |
| CMC | 0.50% | w/v |
| Sodium phosphate monobasic monohydrate | 0.83% | w/v |
| Citric acid monohydrate | 0.17% | w/v |
| Sodium Hydroxide 1N | pH adjust | |
| pH | 5 | |
| Composition 26 | | |
| Sodium chlorite | 1600 | ppm |
| Sodium borate decahydrate | 0.6% | w/v |
| Sodium citrate monohydrate | 0.1% | w/v |

| | |
|---|---|
| pH | 7.6 |

In some embodiments, any of the compositions described herein can be formulated for delivery to the nasopharhynx, trachea and lung tissues, e.g., via pulmonary administration. This delivery can be facilitated via use of a nebulizer or an inhaler. As described herein, the compositions are designed to avoid toxicity to mucous membranes, including the lungs and related tissues. As such, such embodiments are designed to enable successful delivery of a safe dose of chlorine dioxide into the lungs, with subsequent killing of resident viruses and other microbes but with relative sparing of lung tissue. In the case of viral pneumonia, this formulation would be used to reduce the viral load to successfully treat infection. This could be used to treat pneumonia or lung abscess caused by any microbe listed herein, including influenza and coronavirus SARS-CoV-2, responsible for COVID-19. In another embodiment, the compositions described herein could be used to prophylactically treat the lungs after exposure or potential exposures with infected individuals. In another embodiment, the nebulized composition could be delivered to a patient on mechanical ventilation, such as an intubated patient. In another embodiment, a standard or smart inhaler could be used by a patient to deliver medication into the lungs. Dosing could be varied by adjusting the composition nebulized or by varying the flow rate of the nebulized solution.

Aspects of the invention further include use of compositions in accordance with the invention that include sodium chlorite to treat anthrax, including anthrax spores on the eye or in the lungs. Aspects of the invention further include use of compositions in accordance with the invention that include sodium chlorite to treat coccidioidomycosis on the eye or in the lungs. Aspects of the invention further include use of compositions in accordance with the invention that include sodium chlorite to treat west Nile virus on the eye or in the lungs. Aspects of the invention further include use of compositions in accordance with the invention that include sodium chlorite to treat tuberculosis in the eye or in the lungs.

In some embodiments, sodium chlorite or purified sodium chlorite is combined with a steroid. In some embodiments, the steroid component is formulated with the buffer. In some embodiments, the steroid component is formulated with the buffer and a surfactant. In some embodiments the steroid is chosen from the following list: prednisolone acetate, fluorometholone acetate, dexamethasone, fluorometholone, prednisolone phosphate, Dexamethasone phosphate, difluprednate, loteprednol etabonate, loteprednol etabonate, triamcinolone acetonide, betamethasone, hydrocortisone, cortisone. The combination drug can be used to treat blepharitis and dry eye syndrome or any of the infections listed herein, including viral conjunctivitis, EKC, SARS-CoV-2 infection, acanthoemeba conjunctivitis, bacterial keratitis, and fungal keratitis.

In some embodiments, sodium chlorite or purified sodium chlorite is combined with a non-steroidal anti-inflammatory drug (NSAID). In some embodiments, the NSAID is formulated with the buffer. In some embodiments, the NSAID is formulated with the buffer and a surfactant. In some embodiments the NSAID is chosen from the following list: acetylsalicylic acid, diflunisal, salicylamid, indomethacin, diclofenac, sulindac, etodolac, ketorolac, nepafenac, bromfenac, ibuprofen, suprofen, flurbiprofen, naproxen, fenoprofen, ketoprefen, and Celebrex. The combination drug can be used to treat blepharitis and dry eye syndrome or any of the infections listed herein, including viral conjunctivitis, EKC, SARS-CoV-2 infection, acanthoemeba conjunctivitis, bacterial keratitis, and fungal keratitis.

In some embodiments, sodium chlorite, purified sodium chlorite, or the activating buffer is combined or co-formulated with an antihistamine including naphazoline hydrochloride/pheniramine and naphazoline hydrochloride/antazoline phosphate. In some embodiments, sodium chlorite, purified sodium chlorite, or the activating buffer is combined or co-formulated with a mast cell stabilizer including cromolyn or lodoxamide. In some embodiments, sodium chlorite, purified sodium chlorite, or the activating buffer is combined or co-formulated with levocabastine, emedastine (H1 blockers), nedocromil, pemirolast, ketotifen, olopatadine, azelastine, epinastine bepotastine, alcaftadine (mast cell stabilizers and H1 blockers).

In some embodiments, sodium chlorite, purified sodium chlorite, or the activating buffer is combined or co-formulated with cyclosporine to create a targeted therapy for blepharitis and dry eye syndrome. In some embodiments, sodium chlorite or purified sodium chlorite is combined with lifitegrast to create a targeted therapy for keratoconjunctivitis sicca, dry eye syndrome, and/or blepharitis.

In some embodiments, sodium chlorite, purified sodium chlorite, or the activating buffer is combined or co-formulated with cyclosporine to create a targeted therapy for blepharitis and dry eye syndrome. In some embodiments, sodium chlorite, purified sodium chlorite, or the activating buffer is combined or co-formulated with lifitegrast to create a targeted therapy for keratoconjunctivitis sicca, dry eye syndrome, and/or blepharitis.

In some embodiments, sodium chlorite, purified sodium chlorite, or the activating buffer is combined or co-formulated with an anti-viral agent, including, but not limited to the following: idoxuridine, vidarabine, trifluorothymidine, ganciclovir, acyclovir, valacyclovir, penciclovir, famciclovir, or foscarnet.

In some embodiments, sodium chlorite, purified sodium chlorite, or the activating buffer is combined or co-formulated with an anti-biotic agent, including, but not limited to the following: beta-lactams (penicillin with or without beta-lactamase inhibitors), cephalosporins (cefazolin, cefuroxime, ceftazidime, ceftriaxone), monobactams, carbapenems, polymyxin b, bacitracin, vancomycin, aminoglycosides (genatmycin, tobramycin, amikacin, streptomycine, neomycine, paromomycin, kanamycin, spectinomycin), tetracyclines (tetracycline, doxycycline, minocycline, meclocycline), macrolides (erythromycin, azithromycin, clarithromycin, chloramphenicol, clindamycin, fluoroquinolones (ciprofloxacin ofloxacin, norfloxacin, levofloxacin, gatifloxacin, moxifloxacin, besifloxacin.

In some embodiments, sodium chlorite, purified sodium chlorite, or the activating buffer is combined or co-formulated with an anti-fungal agent, including, but not limited to the following: amphotericin B, natamycin, miconazole, ketoconazole, clotrimazole, itraconazole, fluconazole, flucytosine.

In some embodiments, sodium chlorite, purified sodium chlorite, or the activating buffer is combined or co-formulated with an antiamoebic agent, including, but not limited to the following: buguanides (PHMB, chlorhexidine), diamidines (propamidine, hexamidine).

In some embodiments, sodium chlorite, purified sodium chlorite, or the activating buffer is combined or co-formulated with an antiviral agent or agent with anti-infective properties, including, but not limited to the following: vidarabine, trifluridine, acyclovir, famciclovir, valacyclovir, valganciclovir, remdesivir, hydroxychloroquine, chloroquine, azithromycin.

In some embodiments, excipients used include similar excipients to those found in commercially available artificial tears, including Refresh® and Blink® to develop a maximally comfortable formulation that can also help in treating corneal dryness.

Viral conjunctivitis and viral eye infections can be caused by many different types of viruses, including, but not limited to adenovirus (part of the Adenoviridae family consisting of nonenveloped, double-stranded DNA viruses), herpes virus, varicella-zoster virus, picornaviruses, coronaviruses, measles, molluscum contagiosum (pox viruses), epstein-barr virus, coxackie virus, mumps, rubella, smallpox, papovaviruses, togaviruses, orthomyxoviruses including influenza virus, vaccinia, cytomegalo virus and HIV. It should be understood that any virus may cause conjunctivitis, and all virus types are hereby disclosed as potential targets of this composition. It should also be understood that several of the viruses listed can cause other ocular signs, including keratitis, uveitis, retinitis, and endophthalmitis, all of which may be treated via the compositions disclosed in, by either topical, intracameral, intrastromal, subretinal, subtenons, or intravitreal pathways.

Herpetic keratitis caused by the herpes simplex virus is the second most common cause of corneal blindness in the United states after trauma. It can cause conjunctivitis and keratitis with uveitis and necrotizing ulcers. The compositions disclosed herein provide extremely broad-spectrum viral killing without causing ocular toxicity and have been developed to provide broad spectrum viral killing. Herpes zoster ophthalmicus is another cause of significant ocular morbidity with conjunctivitis, keratitis, and uveitis. The compositions disclosed herein can be used to treat HZO and its ocular complications.

Inclusion conjunctivitis is caused by Chlamydia trachomatis. It can lead to trachoma and bilateral keratoconjunctivitis, leading to preventable blindness. The compositions disclosed herein can be used to treat inclusion conjunctivitis.

### Delivery Devices

As disclosed herein, compositions of sodium chlorite include 2 parts that are mixed shortly before use. To accomplish this, several dual chamber delivery systems have been developed, e.g. made up of first and second container, a first container for a sodium chlorite stock solution and a second container for an activating buffer. The containers can have a variety of attachment head configurations, including a male/female luer fitting or a male/female friction fit arrangement that facilitates easy combination and mixing of the 2 products prior to use. Multiple volumes may be employed, including a small volume for ophthalmic clinical procedures where A ranges from 0.3 to 10.0 mL, including 1.5 to 3.0 mL, and B ranges from 0.1 mL to 3.0 mL, including 0.3 to 0.7 mL. In another embodiment, A may range from 10.0 mL to 1000 mL, including 10 ml to 50 mL, or 20 mL to 40 mL. In a larger configuration, B may range from 3.0 mL to 30 mL, including 5.0 ml to 20 mL. It should be understood that A and B can be any volume from 0.3 mL to 1,000 mL, where A refers to the sodium chlorite stock solution container and B refers to the activating buffer container. A and B can also have shapes that are identical or nearly identical, with the exception of the attachment mechanism/dispensing tip. The dispensing tip may allow for dispensing of a drop varying in volume from 10 uL to 150 uL. Any material can be used for the container closure system, including plastics (LDPE, HDPE, polypropylene, and the like), glass, and COP, COC and the like.

Sodium chlorite and purified sodium chlorate are sensitive to UV radiation. Consequently, the container housing sodium chlorite can utilize opaque material. Since opaque material can make it difficult to verify that all the solution in the container is injected from that container into the container holding buffer, a clear material can be used. In that event, foil secondary packaging can be envisioned to provide UV light protection and to limit exposure to air which can affect long term drug stability and evaporation.

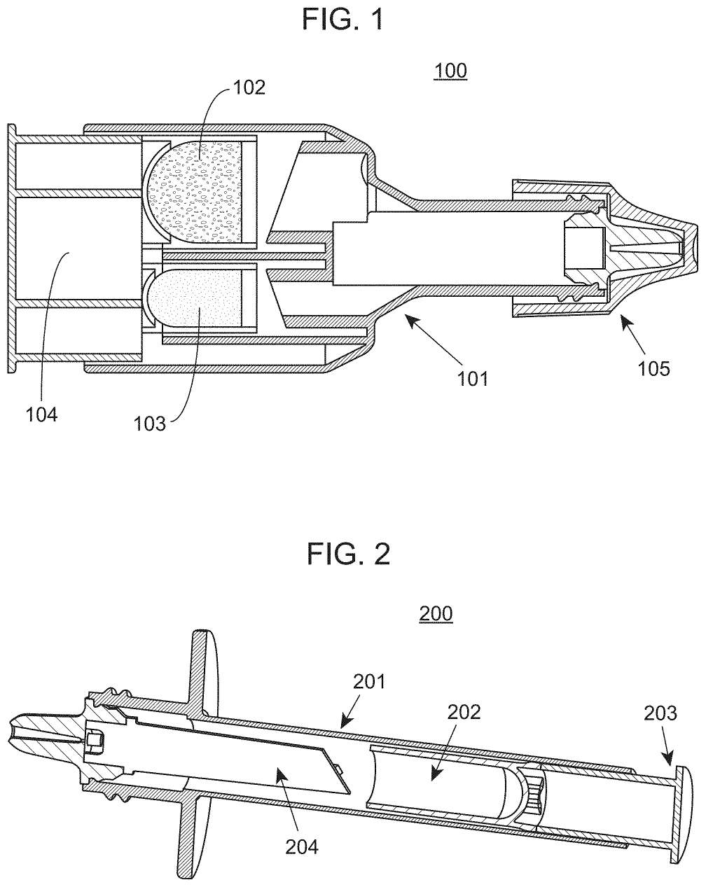

Several embodiments are described below and illustrated in the accompanying Figures. FIG. 1 depicts a delivery device 100 according to an embodiment of the invention. In FIG. 1, injection molded container 101 includes foil-covered vial 102 and foil-covered vial 103. The injection molded container 101 further includes button 104 to push the vials into a rupturing mechanism, causing the fluid to flow into the neck of the device. Luer cap 105 secures the fluid inside the container until dispensing.

FIG. 2 illustrates a delivery device 200 according to an embodiment of the invention. In FIG. 2, injection molded container 201 with clear container 202 is adjacent to button 203. Lancing mechanism 204 is positioned to rupture foil on the clear container 202 and release fluid into the lower part of the container. The neck of the container 202 houses a second fluid or powder.

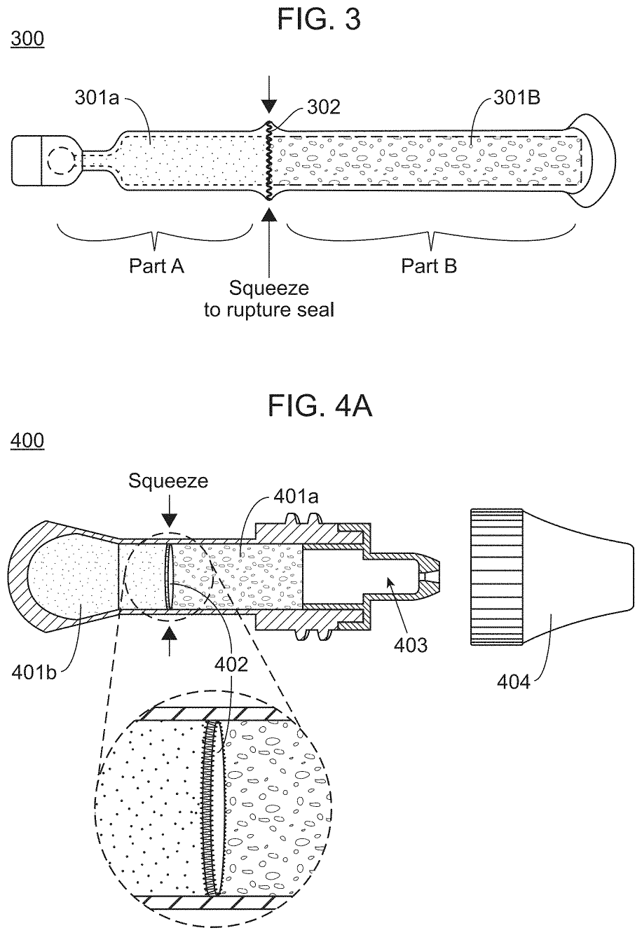

FIG. 3 provides an illustration of another embodiment a delivery device according to the invention. In FIG. 3, blow-fill seal container 300 possesses a rupturable seal 302 between part 301a and part 301b.

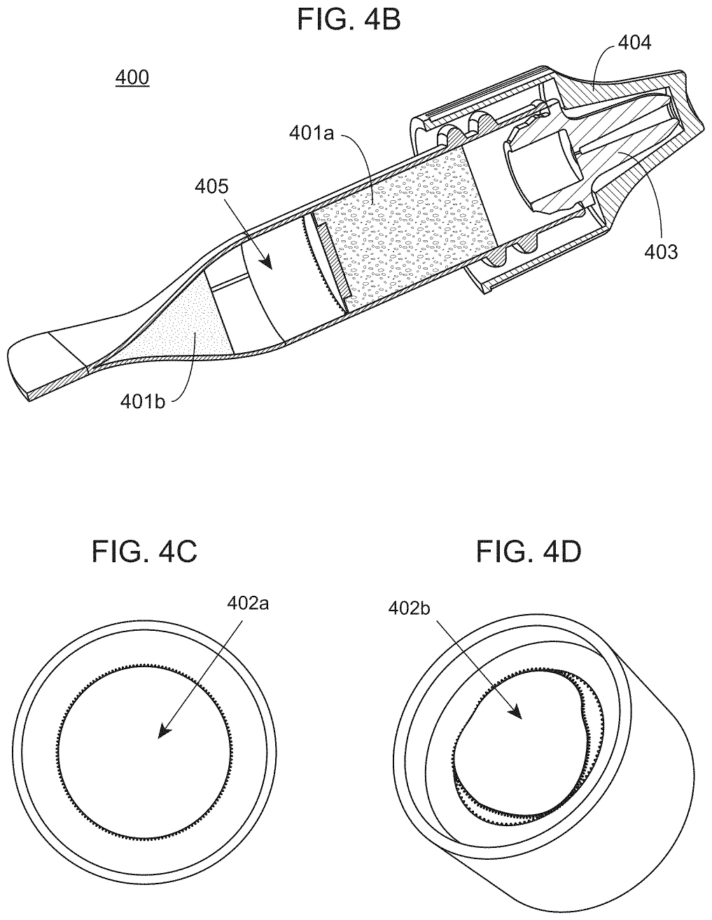

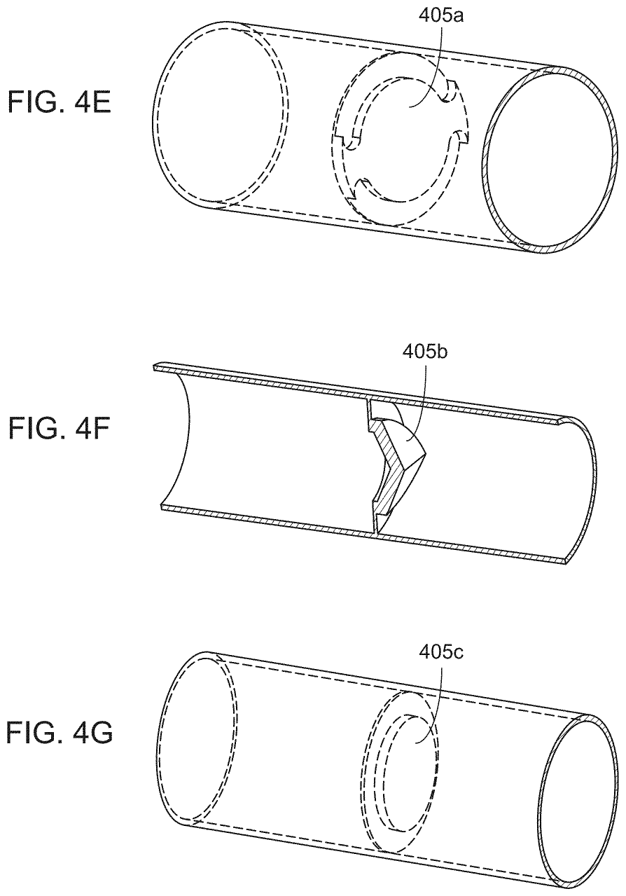

FIGS. 4A to 4D provide illustrations of another embodiment of a delivery device 400 according to the invention. FIG. 4A illustrates an injection molded container having a rupturable seal 402 separating containers 401a and 401b. Luer cap 404 is detached from ophthalmic dispensing tip 403. The central rupturable seal 402 is broken by squeezing. FIG. 4B provides an alternate depiction of delivery device 400 in which with fluid or substance is located within containers 401a and 401b separated by divider 405. The delivery device is closed by luer lock cap 404 that covers ophthalmic dispensing tip 403. FIGS. 4C and 4D provide alternative depictions of seal 402. FIG. 4C illustrates intact seal 402a, while FIG. 4D depicts seal 402b that has ruptured after being squeezed. FIGS. 4E to 4G show alternative divider configurations. FIG. 4E depicts divider 405a having a butterfly divider design that facilitates easy rupturing of seal 402. FIG. 4F depicts divider 405b having a chevron divider design that facilitates ease of rupturing seal 402. FIG. 4G illustrates divider 405c having a standard divider concept.

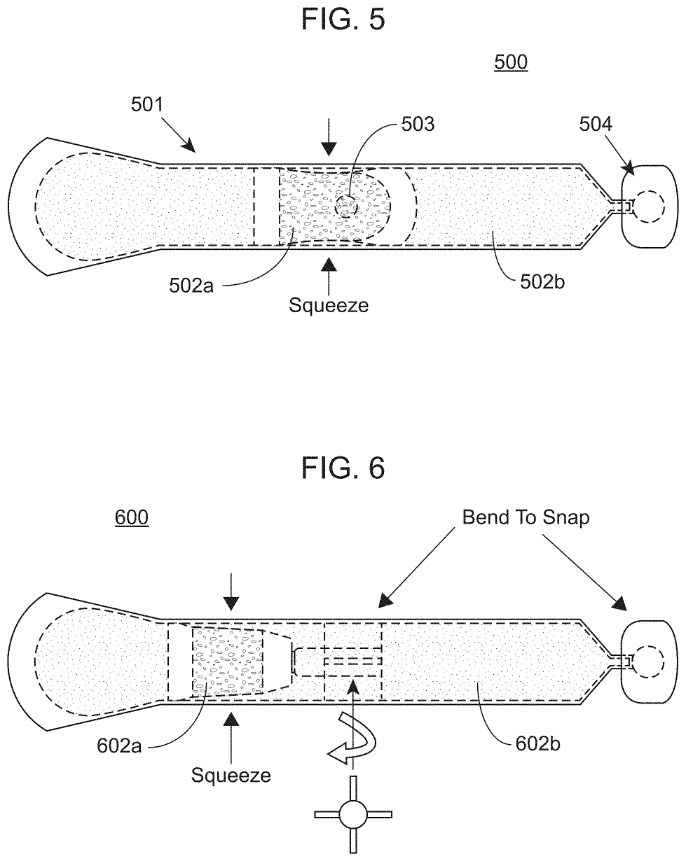

FIG. 5 provides an illustration of delivery device 500 according to one embodiment of the invention. In FIG. 5, injection molded container 501 includes a sealed container 502a placed inside container 502b. Container 501a is ruptured by squeezing and thereby perforating thin membrane 503. The fluids within containers 502a and 502b are subsequently allowed to mix. Tip 504 may be snapped off prior to dispensing the fluid.

FIG. 6 provides an illustration of delivery device 600 according to one embodiment of the invention. In FIG. 6, injection molded container 601 includes parts 602a and 602b. Squeezing and bending results in rupture of part 602a and mixing of the fluids within part 602a and part 602b.

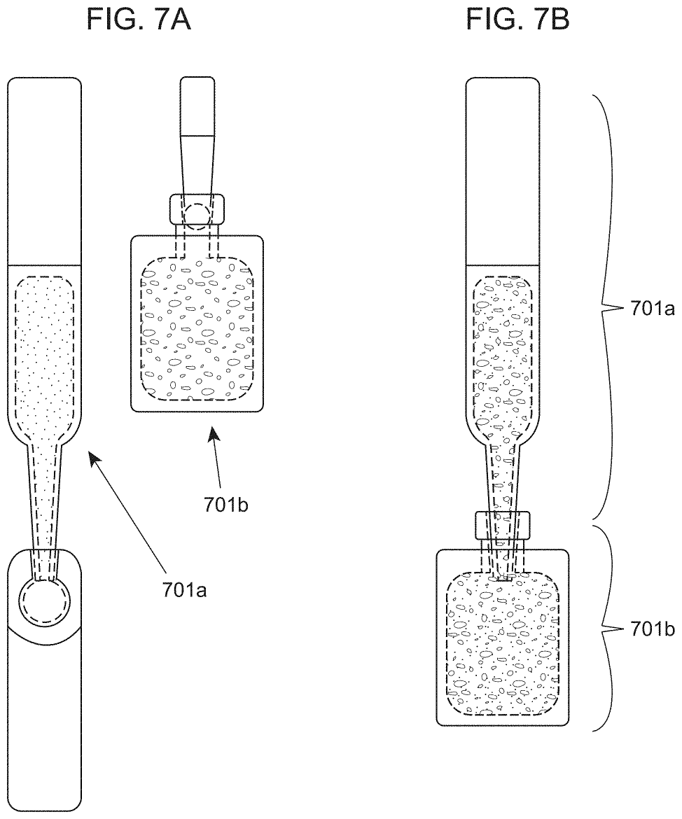

FIGS. 7A and 7B provides an illustration a delivery device according to one embodiment of the invention. As shown in FIG. 7A, the delivery device includes container 701a and container 701b. After removing the container caps, fluid from container 701b is injected into container 701a, allowing easy mixing (FIG. 7B). Fluid can then be dispensed from container 701a into the patient's eye or skin, or different container. Containers can be filled with fluid via any available technology, including blow-fill-seal (BFS). For BFS, containers can be filled side-by-side or filled separately on the same or a different BFS machine.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. E-Beam Terminal Sterilization Testing Results

The stability of purified sodium chlorite following exposure to electron-beam terminal sterilization was tested. Sodium chlorite was placed in low density polypropylene containers (LDPE) and exposed to doses ranging from 10 to 55 kilogray. These results showed that the concentration of sodium chlorite remained stable at doses up to 25 kGy with freezing (sample 12006).

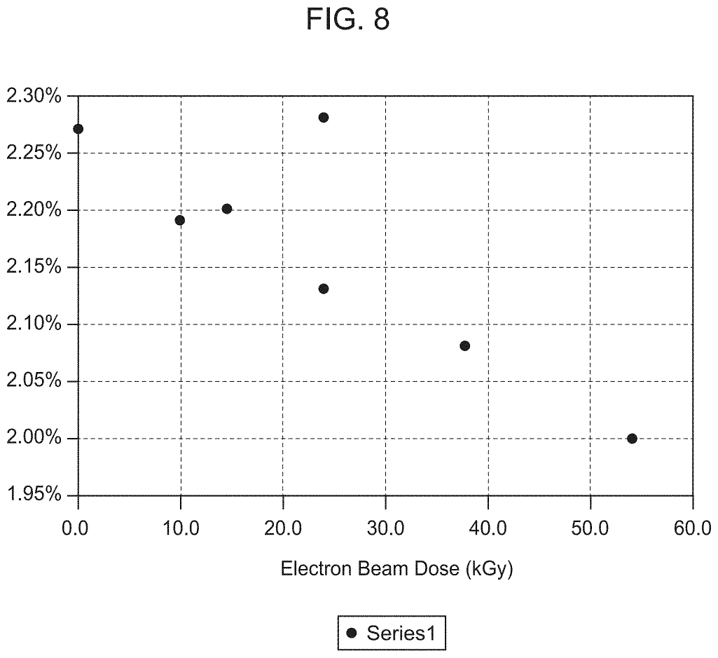

A graphical representation of Sodium Chlorite E-beam testing results demonstrating stability at a number of e-beam doses tested is shown in FIG. 8. FIG. 9 provides Sodium Chlorite E-beam testing parameters and results. Concentrations ranging from approximately 2.10 to 2.30 are considered within the specification.

### II. Adenovirus Kill Studies

The aim of this study was to evaluate the anti-viral efficacy of IRX-101 (purified sodium chlorite combined with a sodium phosphate monobasic monohydrate, citric acid monohydrate, and water buffer) in HeLa cells using an antiviral assay and the colorimetric MTT assay.

Representative images were shown of each condition in infected cells at 72 h post infection. Cell viability was expressed as a percentage of the vehicle treated and uninfected control cells.

### Preparation of Test Compound (IRX-101)

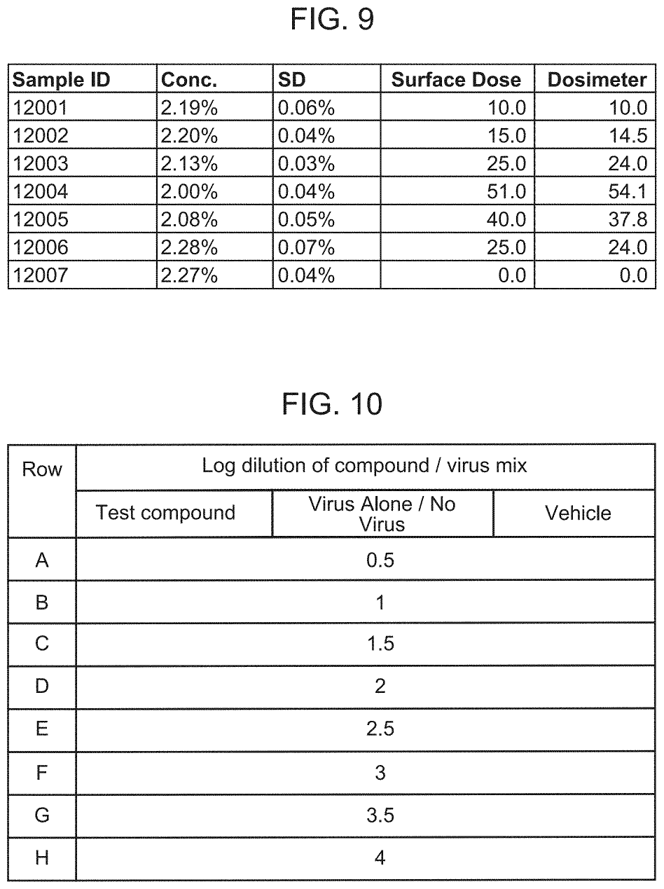

The buffer was prepared by dissolving sodium phosphate monobasic monohydrate (0.25% w/v) and citric acid (0.35% w/v) in water (measured pH=2.69). 10 mL active solution was added to 30 mL of buffer and mixed for 2 minutes. pH was measured at 3.41. FIG. 10 presents a tabular representation of the conditions tested. All conditions were tested in triplicate. Test compound and vehicle were tested with and without Human Adenovirus 5.

### Preparation of Vehicle (Buffer without IRX-101)

10 mL DPBS was added to 30 mL buffer and the pH was measured at ˜2.9. 1M NaOH was used to adjust the pH to match the test compound (measured at 3.39).

### Antiviral Assay

HeLa cells were cultured into test plates according to Charles River SOPs and grown to ˜90% confluency. The neat stock of Human Adenovirus 5 (Adv-5; ATCC VR-5) was incubated with either the test compound or vehicle 1:1 for 2 minutes at room temperature. After 2 minutes of incubation, the virus/compound mix was diluted in an 8-concentration, ½ log dilution scheme. The resulting dilutions were used to infect the HeLa cells in 96-well test plates. A separate dilution was made without Adv-5 to test for cytopathic effects from compound toxicity alone.

Cells were incubated at 37° C./5% CO₂ for 1 hour before addition of overlay, to allow virus to adsorb. After 1 h, the virus mixture was removed, and cells were incubated for 96 h in total and monitored for the appearance of CPE in the control wells. Once CPE was extensive in infected/untreated cells, an MTT assay was performed to quantify cell viability in both infected and uninfected cells.

### RESULTS

### Effect on Uninfected Cells

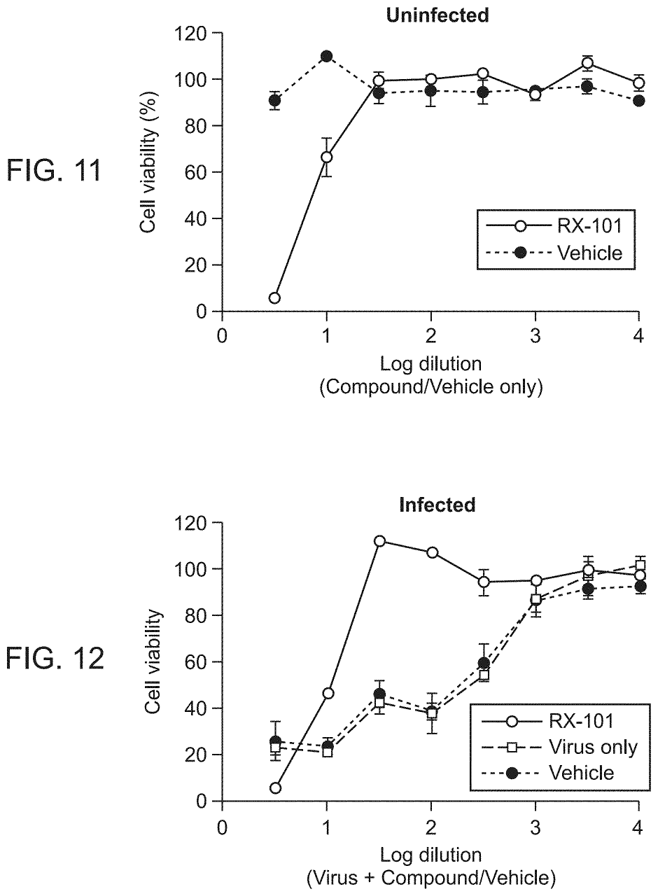

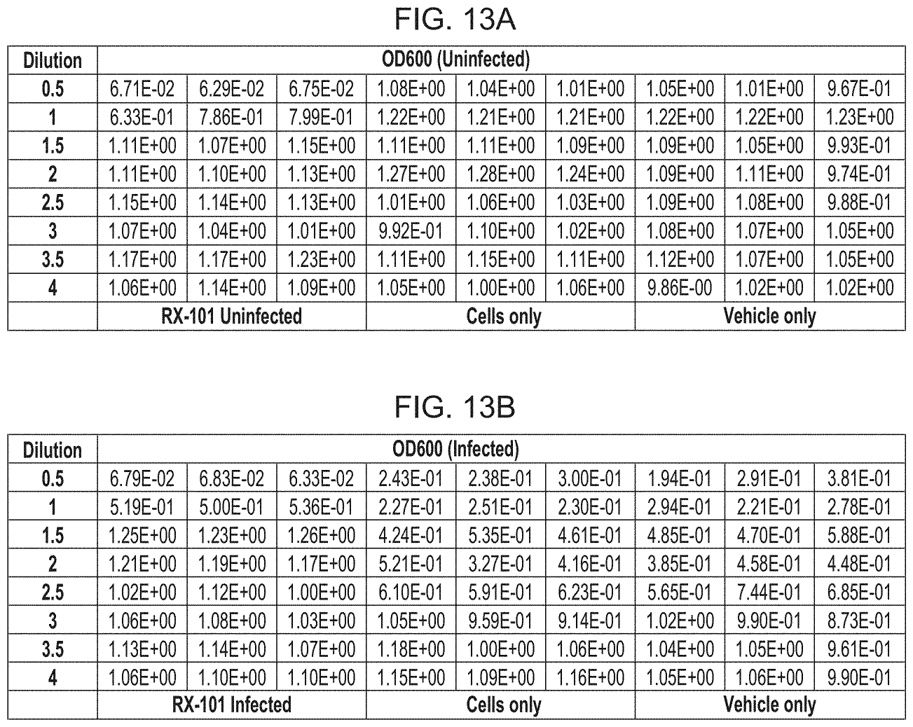

FIG. 11 shows cell viability against concentration of IRX-101/vehicle (expressed as log dilution): Dilution shown is after initial incubation with the blank infection medium. Mean cell viability is plotted±SD (n=3). FIG. 13A depicts the raw data collected for uninfected cell viability.

### Effect on Infected Cells

FIG. 12 shows cell viability against concentration of Adv-5 with either RX101 or vehicle in combination (expressed as log dilution). Dilution shown is after initial incubation with the virus. Mean cell viability is plotted±SD (n=3). Vehicle TCID₅₀ is 2.527. TCID₅₀ for the IRX-101 treated group was not calculated due to no infection. FIG. 13B depicts the raw data collected for infected cell viability.

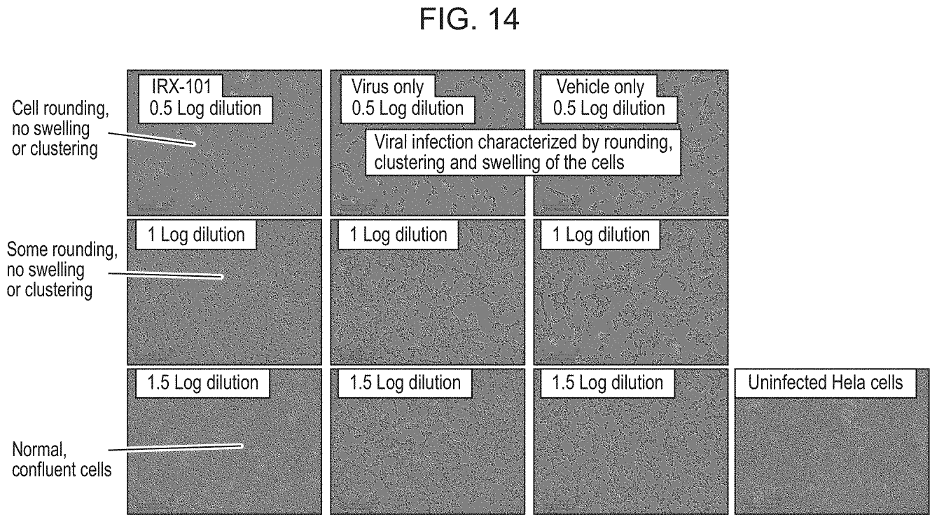

FIG. 14 provides representative images of HeLa cells infected with pre-treated Human Adenovirus-5. Images were taken at 96 hours post infection (hpi). Images of the IRX-101 treated group are similar for both infected and uninfected cells. The IRX-101 treated cells lack the characteristic swelling and clustering typical of infection with Adv-5.

In summary, cells tolerated the vehicle well at all dilutions. Some loss in cell viability was observed at 0.5 and 1 log dilution of IRX-101 with the cells tolerating the compound well at all subsequent dilutions. The range of Human Adenovirus-5 infection was good with cytopathic effects (CPE) and loss of cell viability recorded up to 1:1000 dilution with the virus. Vehicle treatment did not affect the progression of infection. Treatment with IRX-101 appeared to inactivate the virus at concentrations below the CC₅₀ value: Infected cells in the IRX-101 treated group did not show any of the typical Adenovirus-associated CPE (swelling and clustering of cells) compared with vehicle treated, infected cells. The cell viability curve was very similar in the uninfected and infected conditions after treatment with IRX-101. IRX-101 successfully prevented infection of Human Adenovirus-5 virus in HeLa cells compared with vehicle treated, infected cells. Treatment with the vehicle, adjusted to the same pH, had no effect on the infectivity of the virus. These assays demonstrate the efficacy of IRX-101 to kill adenovirus, providing evidence of the clinical value of this compound to kill adenovirus infecting the ocular surface. These data, combined with the excellent ocular safety data of the tested sodium chlorite and buffer compound, suggest that this compound and derivative described herein, may be used to treat viral conjunctivitis, including adenoviral conjunctivitis.

### III. SARS-CoV-2 Kill Studies

### OBJECTIVES

The objective of this study was to test chemical formulations as an eye wash, against SARS-CoV2-infected Vero E6 cells as determined by plaque reduction assays.

### MATERIALS AND METHODS

### Buffer Preparation

To prepare a part 1 buffer (per Irenix protocol), 0.25 g of sodium phosphate monobasic monohydrate and 0.35 g of citric acid monohydrate were dissolved in 100 mL of distilled water, and filter sterilized (Final pH=2.72; Filter sterilized (0.22 um)). A part 2 buffer included an active solution provided by Irenix (NaClO₃ in solution, solvent dH2O, storage RT). The buffers of parts 1 and 2 were combined at a ratio of 3:1 (part 1:2; pH=3.3) and a ratio of 1:1 (part 1:2; pH=3.3). A part 3 buffer (control buffer per Irenix protocol) was made using the same protocol as for Part 1. However, this buffer was titrated with 1M sodium hydroxide to increase its pH to 4.0. The final pH was 3.4. The solution was filter sterilized (0.22 um).

The part 1 and 2 buffers and the active solution (Purite, provided with iRenix) were stored at ambient temperature in the dark. The active solution and buffer solution (sodium hydroxide-titrated) were mixed in a 3:1 dilution. The solution was stable for four hours and thus a dilution was freshly made prior to executing the assay. A part 4 buffer included 2% DMEM as a control (to represent normal infection conditions).

### Cell Preparation

The cell line utilized for the plaque reduction assay was Vero E6 cells (ATCC® CRL-1586). These cells are grown from a frozen aliquot of a laboratory working cell line. Passage number is limited to no more than 50 passages from the original aliquot. Cells are grown in T150 flasks in 1×DMEM (ThermoFisher cat. no. 12500062) supplemented with 2 mM L-glutamine (Hyclone cat. no. H30034.01), non-essential amino acids (Hyclone cat. no. SH30238.01), and 10% heat inactivated Fetal Bovine Serum (FBS) (Atlas Biologicals cat. no. EF-0500-A).

On the day previous to executing the assay, Vero E6 cells were removed from T150 flasks by trypsinization (0.25% Trypsin, Corning cat. no. 25-053-CI) and measured for count and viability by Hemocytometer in trypan blue. Cells were resuspended to 6×10⁵ cells per mL in 1×DMEM (supplemented as indicated above) and plated at 1 mL per well (600,000 cells/well) in 12 well plates. The plates were then incubated for approximately 24 hours to allow cell adherence at 37° C., 5% CO₂.

### Cell Staining

48 hours post virus infection and plaque assay, cells were stained with 1×PBS and neutral red solution (NRS) (0.33% NRS, Sigma Aldrich cat. no. N2889) at a ratio of 11.5 mL 1×PBS and 0.5 mL NRS per 12-well plate (1 ml/well). Staining was carried out overnight (˜12 hrs).

### Viral Preparation

The virus strain used for the assay was SARS-CoV2, USA WA 01/2020, CSU V2 03/17/202 passage 3. Virus stocks were obtained from BEI Resources and amplified in Vero E6 cells to Passage 3 (P3) with a titer of 3.4×10⁶ PFU/mL. Stocks were stored at −80° C. Table 9, below, presents the plate format.

TABLE 9
Plate Format

| | A | B | C |
|---|---|---|---|
| 1 | Part 1 + 2 and | Part 3 and | 1XPBS or |
| | 10⁻¹ virus | 10⁻¹ virus | 1XDMEM + 10⁻¹ virus |
| 2 | Part 1 + 2 and | Part 3 and | 1XPBS or |
| | 10⁻² virus | 10⁻² virus | 1XDMEM + 10⁻² virus |
| 3 | Part 1 + 2 and | Part 3 and | 1XPBS or |
| | 10⁻³ virus | 10⁻³ virus | 1XDMEM + 10⁻³ virus |
| 4 | Part 1 + 2 and | Part 3 and | 1XPBS or |
| | 10⁻⁴ virus | 10⁻⁴ virus | 1XDMEM + 10⁻⁴ virus |

### Assay Setup

There were three replicates per dilution sample. Each well contained 200 uL of the final diluted solution. 300 uL of virus stock was diluted into 2.7 mL of 1×PBS or 1×DMEM, which resulted in a 10⁻¹ virus dilution. 600 uL or 200 uL of the 10⁻¹ virus dilution was transferred into 200 uL of the respective formulations (Part 1+2, Part 3, 1×PBS or 1×DMEM), depending on either having a 3:1 or 1:1 virus-formulation dilution. The 3:1 dilution (virus:active solution) included 600 uL (10⁻¹ dilution)+200 uL of active solution or controls. The 1:1 dilution (virus:active solution) included 200 uL (10⁻¹ dilution)+200 uL of active solution or controls. Samples were incubated at ambient temperature for 2 minutes. Serial dilutions were done by transferring 120 uL of the solutions from step 2 into 1080 uL of IXDMEM. This step was repeated until 10⁻⁴ dilution was reached. 200 uL of corresponding solution was dispensed into corresponding wells. Plates were incubated at ambient temperature on a rocker for 1 hour to allow the virus to adsorb. After an hour, wells were overlaid with 2% agarose and 2×DMEM (supplemented with 10% FBS). 48 hours after adding the overlay, wells were stained with neutral red solution in 1×PBS and incubated at 37° C./5% CO₂ for 12 hr prior to counting plaques

### RESULTS

All figures were graphed using Graph Prism Ver.8.3.1. For each figure, a one-way ANOV, with multiple comparisons test was conducted with all treatments compared to the control and 1×DMEM groups.

Virus titer was evaluated at 48 hours post infection and compared to a 1×DMEM control. There was a higher plaque reduction efficacy in samples when the virus was diluted with 1×PBS versus 1×DMEM to obtain the 10⁻¹ stock. These data suggest that the components in 1×DMEM may interfere with the efficacy with which the active solution inactivates the virus. Complete inactivation of the virus was observed following incubation with the active solution for all experiments when the diluent was 1×PBS.

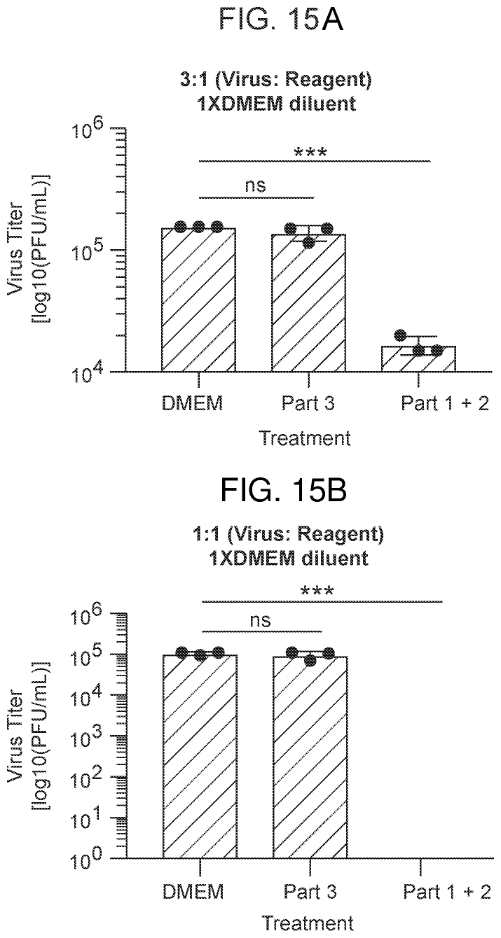

FIGS. 15A-B depict the results of a plaque reduction assay with 1×DMEM as a diluent. Treatment efficacy was measured 48 hours post infection via plaque reduction assay. The virus was diluted in 1×DMEM. 3:1 (FIG. 15A) and 1:1 (FIG. 15B) refers to the solutions used to treat the virus. The controls are 1×DMEM and Part 3. Active solution is Part 1+2. Fold change was calculated compared to the 1×DMEM control using the one-way ANOVA multiple comparisons test (ns=not significant, ***=p<0.0002, ****=p<0.0001).

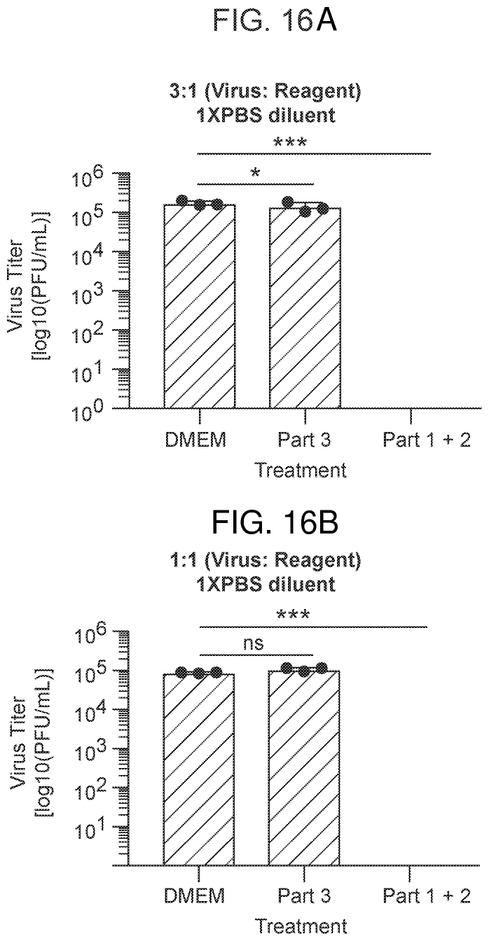

FIGS. 16A-B depict the results of a plaque reduction assay with 1×PBS as a diluent. Treatment efficacy was measured 48 hours post infection via plaque reduction assay. The virus was diluted in 1×PBS. 3:1 (FIG. 16A) and 1:1 (FIG. 16B) refers to the solutions used to treat the virus. The controls are 1×DMEM and Part 3. The active solution is Part 1+2. Fold change was calculated compared to the 1×DMEM control using the one-way ANOVA multiple comparisons test (ns=not significant, *=p<0.0275, ***=p<0.0002, ****=p<0.0001).

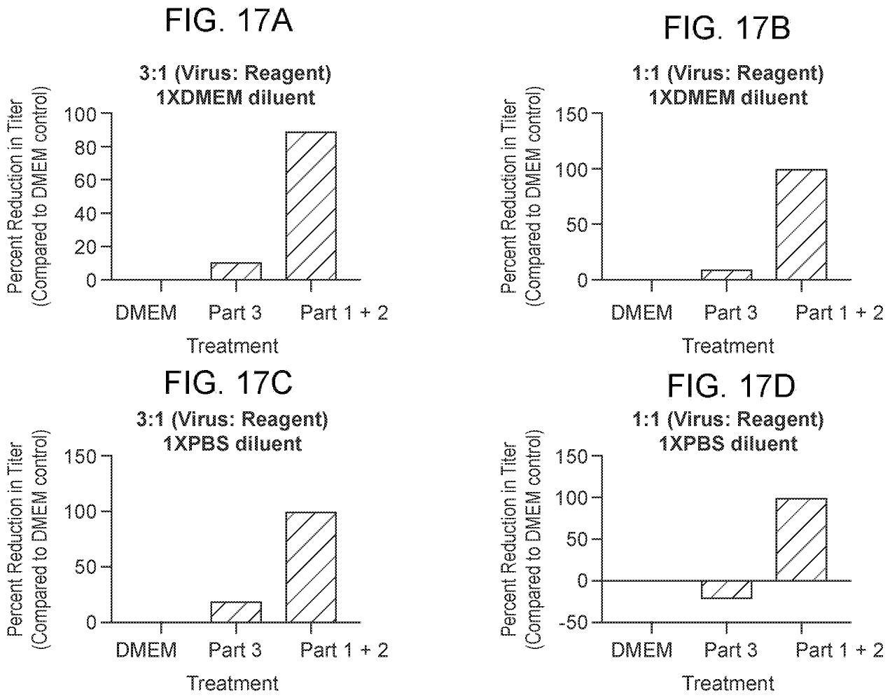

FIG. 17A-D shows percent reduction of PFU/mL in virus exposed to Part 1+2 (sodium chlorite composition), Part 3 (buffer control, pH adjusted), and DMEM viral culture media. 3:1 and 1:1 refers to the solutions used to treat the virus. Part 1+2 demonstrates complete PFU reduction in 3 of the 4 test scenarios, with approximately 90% reduction in the fourth condition (DMEM diluent, 3:1 dilution)

### CONCLUSIONS

These results show a robust anti-viral response of SARS-CoV-2 to the formulation of purified sodium chlorite and buffer, mixed shortly before use. It was concluded that the Part 1+2 formulation is highly effective at killing SARS-CoV-2, and, combined with its excellent ocular safety profile as demonstrated in previous GLP toxicity studies, has promise in treating ocular SARS-CoV-2, including conjunctivitis and other side effects of infection, including chemosis, injection, tearing, ocular pain, corneal involvement, episcleral, scleral involvement, and SARS-CoV-2 virus and viral particles in tears. This complete kill was demonstrated even in the face of 3 to 1 virus to compound dilution, showing the strength of the viral kill. Compounds disclosed herein can be used as a drop, mist, salve, gel, or ointment to apply to the human eye to prevent or treat ocular SARS-CoV-2 infection. The chemical compounds disclosed herein are expected to have similar efficacy in killing all types of coronavirus given their similar structure, including mutated forms of the SARS-CoV-2 virus that may occur in the coming months and years. This formulation can used to prevent or treat SARS-CoV-2 infection before symptoms are present, such as immediately prior to or following exposure to potentially infected individuals.

### REFERENCES

1. Wu P, Duan F, Luo C, et al. Characteristics of Ocular Findings of Patients With Coronavirus Disease 2019 (COVID-19) in Hubei Province, China. JAMA Ophthalmol 2020.
2. Seah I, Agrawal R. Can the Coronavirus Disease 2019 (COVID-19) Affect the Eyes? A Review of Coronaviruses and Ocular Implications in Humans and Animals. Ocul Immunol Inflamm 2020; 28(3):391-5.
3. Lu CW, Liu X F, Jia Z F. 2019-nCoV transmission through the ocular surface must not be ignored. Lancet 2020; 395(10224):e39.
4. Colavita F, Lapa D, Carletti F, et al. SARS-CoV-2 Isolation From Ocular Secretions of a Patient With COVID-19 in Italy With Prolonged Viral RNA Detection. Ann Intern Med 2020.
5. Li J O, Lam D S C, Chen Y, Ting D S W. Novel Coronavirus disease 2019 (COVID-19): The importance of recognising possible early ocular manifestation and using protective eyewear. Br J Ophthalmol 2020; 104(3):297-8.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of treating a subject for a microbial eye condition, the method comprising:
administering to the subject an activated sodium chlorite composition comprising:
(a) sodium chlorite; and
(b) a buffer component prepared from sodium phosphate monobasic monohydrate and citric acid;
wherein the composition has a pH ranging from 3 to 6 and is substantially free of heavy metals; to treat the subject for the microbial eye condition.

2. The method according to claim 1, wherein the sodium chlorite is present in an amount ranging from 100 to 10,000 ppm.

3. The method according to claim 2, wherein the sodium chlorite is present in an amount ranging from 4,000 to 6,000 ppm.

4. The method according to claim 1, wherein the composition has a pH ranging from 3.3 to 7.5.

5. The method according to claim 4, wherein the composition has a pH of 3.0 to 4.5.

6. The method according to claim 4, wherein the composition has a pH of 4.5 to 5.5.

7. The method according to claim 6, wherein the buffer component is prepared from sodium phosphate monobasic monohydrate, citric acid, and sodium hydroxide.

8. The method according to claim 1, wherein the buffer component comprises sodium phosphate monobasic monohydrate in an amount ranging from 0.05% w/v to 0.95% w/v.

9. The method according to claim 1, wherein the buffer component comprises citric acid in an amount ranging from 0.05% w/v to 1.5% w/v.

10. The method according to claim 1, wherein the amount of heavy metals in the composition, if present, is 2.0 ppm or less.

11. The method according to claim 1, wherein the composition comprises 400 ppm or less chlorate ion.

12. The method according to claim 1, wherein the composition further comprises a surfactant.

13. The method according to claim 1, wherein the method comprises administering the composition to an eye of the subject.

14. The method according to claim 1, wherein the subject is a human.

15. The method according to claim 1, wherein the microbial eye condition is conjunctivitis.

16-71. (canceled)

72. A delivery device comprising:
(a) a first container comprising a sodium chlorite stock solution; and
(b) a second container comprising an activating buffer;
wherein the device is configured to combine the sodium chlorite stock solution and activating buffer to produce an activated sodium chlorite composition and administer the activated sodium chlorite composition to a subject.

73-82. (canceled)

83. The delivery device according to claim 72, wherein the stock solution and the activating buffer are not aseptically introduced into the first and second containers, respectively, and the first and second containers are terminally sterilized.

84. The delivery device according to claim 83, wherein the first and second container are terminally sterilized with e-beam terminal sterilization.

85. (canceled)

86. The delivery device according to claim 72, wherein the sodium chlorite stock solution is protected from UV degradation by an opaque barrier.

87. The delivery device according to claim 72, wherein the sodium chlorite stock solution is protected from UV degradation by secondary foil packaging.
